(19) United States
(12) Patent Application Publication
(10) Pub. No.: US 2019/0374454 A1
(43) Pub. Date: Dec. 12, 2019

(54) OXYBENZONE-FREE COMPOSITIONS

(71) Applicant: SYMRISE AG, Holzminden (DE)

(72) Inventors: William JOHNCOCK, Reinbek (DE); Martina ISSLEIB, Hoisdorf (DE)

(21) Appl. No.: 16/004,839

(22) Filed: Jun. 11, 2018

(51) Int. Cl. A61K 8/37; A61K 8/35; A61K 8/34; A61K 8/41; A61K 8/49; A61K 8/55; A61K 8/27; A61K 8/29; A61K 8/06; A61K 8/92; A61K 9/00; A61Q 5/00; A61Q 17/02; A61Q 17/04

(52) U.S. Cl. CPC A61K 8/37; A61K 2800/74; A61K 8/345; A61K 8/347; A61K 8/41; A61K 8/4946; A61K 8/55; A61K 8/27; A61K 8/29; A61K 8/062; A61K 8/064; A61K 8/922; A61K 9/0014; A61Q 5/00; A61Q 17/02; A61Q 17/04; A61K 2800/26; A61K 2800/43; A61K 2800/51; A61K 2800/522; A61K 2800/524; A61K 8/35

(57) ABSTRACT

Provided herein are compositions comprising at least 3.5% Diethylhexyl 2,6-Naphthalate by weight, wherein the composition is a cosmetic, dermatological or pharmacological composition for protection of human skin and/or human hair against UV radiation and wherein the compositions is free of Oxybenzone.

### FIELD OF INVENTION

The present invention relates to cosmetic, dermatological or pharmacological compositions for protection of the human skin and human hair against the effects of ultraviolet (UV) radiation and methods for obtaining them.

### STATE OF THE ART

UV absorbers are compounds which have a pronounced absorption capacity for ultraviolet radiation. They are used in particular as sunscreens in cosmetic, dermatological and pharmacological preparations, but also to improve the light fastness of industrial products, such as paints, varnishes, plastics, textiles, polymers such as, for example, polymers and copolymers of mono- and di-olefins, polystyrenes, polyurethanes, polyamides, polyesters, polyureas and polycarbonates, packaging materials and rubbers.

UV rays are classified according to their wavelength as UVA rays (320-400 nm, UVA-I: 340-400 nm, UVA-II: 320-340 nm) or UVB rays (280-320 nm). UV rays can cause acute and chronic damage to the skin, the type of damage depending on the wavelength of the radiation. For instance, UVB radiation can cause sunburn (erythema) extending to most severe burning of the skin. Reduction in enzyme activities, weakening of the immune system, disturbances of the DNA structure and changes in the cell membrane are also known as harmful effects of UVB rays. UVA rays penetrate into deeper layers of the skin where they can accelerate the aging process of the skin. The shorter wave UVA-II radiation additionally contributes to the development of sunburn. Moreover, UVA radiation can trigger phototoxic or photo allergic skin reactions. Very frequent and unprotected irradiation of the skin by sunlight leads to a loss of skin elasticity and to increased development of wrinkles. In extreme cases, pathogenic changes in the skin extending to skin cancer are observed. To attenuate these negative effects of UV radiation, materials which absorb or reflect UV light, generally called UV absorbers, are used in cosmetic, dermatological and pharmacological preparations. The UV absorbers are classified as UVA and UVB absorbers depending on the location of their absorption maxima; if a UV absorber absorbs both UVA and UVB, it is referred to as a UVA/B broadband absorber.

The degree of efficacy of cosmetic, dermatological and pharmacological preparations for protection of the human skin from the erythema which is induced by UV radiation is determined by their Sun Protection Factor (SPF), which is the ratio of the energy required to show the first defined redness (erythema) of human skin which has been protected to the energy required to show the first defined redness of human skin which has not been protected. The amount of energy required to the first signs of erythema on human skin of Fitzpatrick classification 2 (light Caucasian) is 200 J/m2 which is also known as the Minimal Erythemal Dose (MED). Natural sunlight at 40° N on a clear day will deliver this dose in about 10 minutes. So a cosmetic, dermatological or pharmacological preparation for the protection of the human skin from the erythema with an SPF of 50 will theoretically protect skin from burning for 500 minutes. In addition to the SPF most regulatory authorities also stipulate that the formulations designed to protect human skin from erythema should also have sufficient absorption in the UVA range of the spectrum.

In order to achieve the desired protection from UV radiation cosmetic, dermatological and pharmacological preparations contain a mixture of UV filters with varying concentrations and the choice of UV filters used is determined by the legislation within the country or economic area. For example UV filters which can be used for the protection of skin are regulated in the USA by the America FDA via their OTC monograph system and are regulated in the European Union by the Cosmetic Regulation. Regulations covering the use of UV filters exist in other countries and regions as well. These regulations not only stipulate the filters which can be used but also fix a maximum usage level for each UV filter. So if a particular UV filter which is on the regulated lists is subject to questions about its suitability for use because of perceptions about its safety or effect on the environment, there is public pressure for this particular product to be replaced, even if the regulatory authorities have deemed the product to be safe. In order to maintain the desired protection against UV radiation, this replacement can only normally be done by use of another UV filter which is on the regulated list.

One of the UV filters under pressure to be removed from formulations is Benzophenone-3, also known under its USAN designation Oxybenzone. Generally, the use of Oxybenzone is questionable (https://www.ewg.org/skindeep/ingredient/.../OXYBENZONE). This has led to the need to find a suitable replacement, particularly in the United States of America where the use of Benzophenone-3 in sun care cosmetic, dermatological and pharmacological preparations for protection of the human skin and human hair against the harmful effects of ultraviolet (UV) radiation is very common, particularly in formulations with a high efficacy with an SPF of 30 or more.

A typical SPF 50+ formulation in the USA uses the maximum allowed concentrations of the UV filters Avobenzone, Octisalate, Homosalate, Octocrylene, and Oxybenzone. Therefore the replacement of the problematic Oxybenzone is a major challenge. Some formulations use the presence of so called SPF boosters to increase the SPF. One example is the ingredient Sunspheres™ (INCI Styrene/Acrylates copolymer) which are insoluble polymer particles which reflect UV radiation. The use of this ingredient leads to increased white residues of the formula being left on the skin. Other examples of SPF boosters are the use of esters which also have a UV absorbing chromophore such as Butyloctyl Salicylate, Polycrylene and, Ethylhexylmethoxycrylene. When these are used in amounts greater than 3% then they would probably have an SPF and will be in conflict with the FDA definition of a UV filter, so their boosting potential is minimised by the requirement that they be used in sufficiently low quantities so as not to generate an SPF when used alone.

Therefore, it was an object of the present invention to provide an UV-filter which can replace Oxybenzone and which can replace Oxybenzone in cosmetic, dermatological and pharmacological compositions and simultaneously maintain the same SPF as the compositions with Oxybenzone.

### SUMMARY OF THE INVENTION

This object is solved by the independent claims of the present invention. Preferred embodiments are part of the dependent claims.

One object of the present invention is a composition comprising at least 3.5% Diethylhexyl 2,6-Naphthalate by weight, wherein the composition is a cosmetic, dermatological or pharmacological composition for protection of human skin and/or human hair against UV radiation and wherein the compositions is free of Oxybenzone.

The terms Oxybenzone and Benzophenone-3 are used simultaneously in the application and also refer, as explained above, to the same substance.

2,6-Diethylhexylnaphthalate is well known as a photo stabilizer of Avobenzone (U.S. Pat. No. 6,126,925, Bonda et. al). U.S. Pat. No. 612925 does teach that 2,6-Diethylhexylnaphthalate can increase the SPF of formulations containing Avobenzone, Octisalate, Octinoxate and Oxybenzone to a surprisingly high SPF without precisely stipulating the SPF obtained, nor the combination of UV filters used. Bonda provided more information in the article published in Cosmetic & Toiletries (2000, volume 115, No. 6 pages 37-45) in which in-vivo SPF measurements of UV filter combinations were shown.

U.S. Pat. No. 6,444,195 (Cole et al) teaches the use of the combination of Avobenzone with 2,6-Diethylhexylnaphthalate and Oxybenzone to achieve photostable sun care formulations with an SPF of 20 or more, indicating again that Oxybenzone is required to obtain higher SPFs.

U.S. Pat. No. 7,204,973 and U.S. Ser. No. 01/026,790 (Beiersdorf) describes 2,6-Diethylhexylnaphthalate as a solvent for solid UV filters.

U.S. Pat. No. 7,204,975 (Beiersdorf) describes 2,6-Diethylhexylnaphthalate to improve the dispersion of particulate UV filters such as titanium and zinc oxides.

U.S. Pat. No. 6,491,901B2 (Beiersdorf) describes 2,6-Diethylhexylnaphthalate in combination with a maximum amount of 3.5% Octocrylene to improve the photostabilty of Avobenzone.

U.S. Pat. No. 7,214,365 B2 (Beiersdorf) describes 2,6-Diethylhexylnaphthalate to protect photolabile hydrophilic substances from degradation.

US2004247540A1 (Beiersdorf) describes 2,6-Diethylhexylnaphthalate in combination with water soluble UV filters to care for the skin and prevent it from drying out on exposure to UV radiation.

U.S. Pat. No. 7,201,893B2 (Beiersdorf) describes 2,6-Diethylhexylnaphthalate in combination with phos-phate or sulfate based emulsifiers to improve the photostability of Avobenzone.

US2004170660A1 (Beiersdorf) describes 2,6-Diethylhexylnaphthalate to improve the physical stability of formulations, including suncare formulations containing insect repellents.

U.S. Pat. No. 7,244,417B2 (Beiersdorf) describes 2,6-Diethylhexylnaphthalate in combination with waxes and oils to improve the photostability of Avobenzone and other photolabile ingredients.

U.S. Pat. No. 7,204,974B2 (Beiersdorf) describes 2,6-Diethylhexylnaphthalate in combination with lipids having a polarity of less than 30 mN/m to improve the photostability of Avobenzone and other photolabile ingredients.

The inventors have surprisingly found that using the ester 2,6-Diethylhexylnaphthalate, sold by the company Symrise under the tradename of Corapan® TQ can replace Oxybenzone in cosmetic, dermatological and pharmacological preparations for protection of the human skin and maintain the same SPF as the formulations with Oxybenzone.

### FIGURES

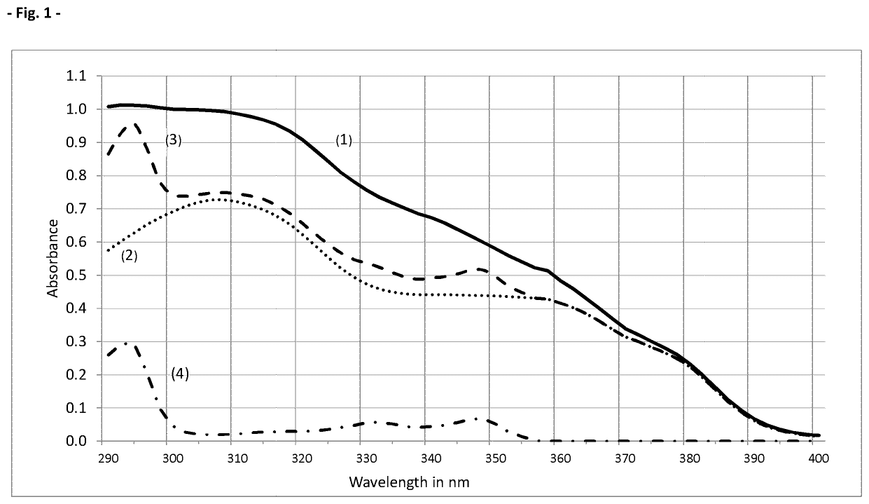

FIG. 1 shows the absorbance curves of of 10 mg/l of the following combinations of UV filters in solution:
(1) 9.0% by weight of Octocrylene+9.0% by weight of Homosalate+5.0% by weight of Octisalate+2.3% by weight of Avobenzone+6.0% by weight of Oxybenzone;
(2) 9.0% by weight of Octocrylene+9.0% by weight of Homosalate+5.0% by weight of Octisalate+2.3% by weight of Avobenzone;
(3) 9.0% by weight of Octocrylene+9.0% by weight of Homosalate+5.0% by weight of Octisalate+2.3% by weight of Avobenzone+10% by weight of Diethylhexyl Naphthalate; and
(4) 10% Diethylhexyl Naphthalate.

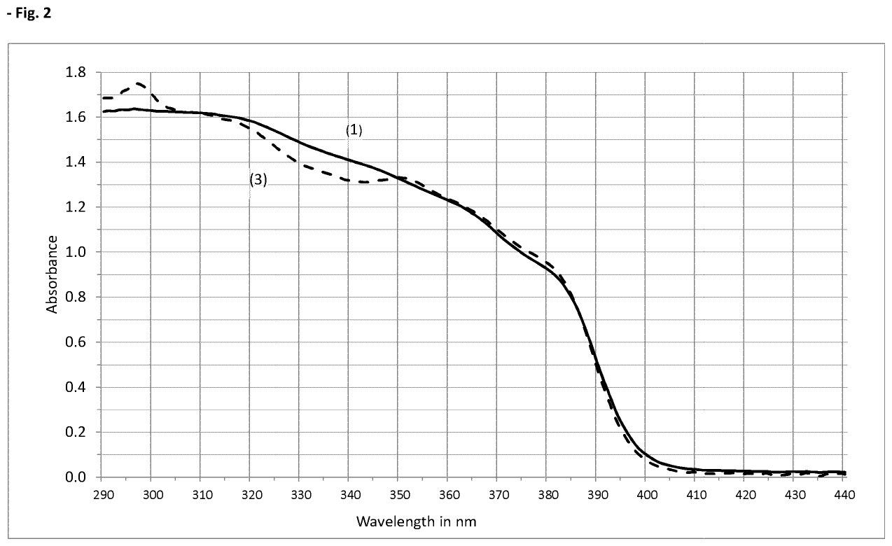

FIG. 2 shows the absorbance curves of a thin film (1.3 mg/cm2) of the following emulsions (also shown in Table 1 of the present invention):
(1) 9.0% by weight of Octocrylene+9.0% by weight of Homosalate+5.0% by weight of Octisalate+2.3% by weight of Avobenzone+6.0% by weight of Oxybenzone; and
(3) 9.0% by weight of Octocrylene+9.0% by weight of Homosalate+5.0% by weight of Octisalate+2.3% by weight of Avobenzone+10% by weight of Diethylhexyl Naphthalate.

### DETAILED DESCRIPTION OF THE INVENTION

In some embodiments according to the invention the composition further comprises UV filters. Suitable UV filters are, for example, organic UV absorbers from the class of 4-aminobenzoic acid and derivatives, salicylic acid derivatives, benzophenone derivatives, dibenzoylmethane derivatives, diphenylacrylates, 3-imidazol-4-ylacrylic acid and its esters, benzofuran derivatives, benzylidenemalonate derivatives, polymeric UV absorbers containing one or more organosilicon radicals, cinnamic acid derivatives, camphor derivatives, trianilino-s-triazine derivatives, 2-hydroxyphenylbenzotriazole derivatives, menthyl anthranilate, benzotriazole derivatives and indole derivatives.

Specific UV filters which can be used are for example as follows: UVB filters:
p-aminobenzoic acid
ethyl p-aminobenzoate (25 mol) ethoxylated
2-ethylhexyl p-dimethylaminobenzoate
homomenthyl salicylate (homosalate) (Neo Heliopan [symbol] HMS)
2-ethylhexyl salicylate (Neo Heliopan® OS)
triethanolamine salicylate (Neo Heliopan® TS)
menthyl anthranilate (Neo Heliopan® MA)
2-ethylhexyl p-methoxycinnamate (Neo Heliopan® AV)
isoamyl p-methoxycinnamate (Neo Heliopan® E 1000)
2-phenylbenzimidazole sulfonic acid (Neo Heliopan® Hydro) and its salts
3-(4′-trimethylammonium)benzylidenebornan-2-one methyl sulphate
3-(4′-sulpho)benzylidenebornan-2-one and salts
3-(4′-methylbenzylidene)-d,l-camphor (Neo Heliopan® MBC)
N-[(2 and 4)-[2-(oxoborn-3-ylidene)methyl]benzyl]acrylamide polymer
4,4′-[(6-[4-(1,1-dimethyl)aminocarbonyl)phenylamino]-1,3,5-triazine-2,4-diyl)diimino]bis(benzoic acid 2-ethylhexyl ester) (Uvasorb® HEB)
benzylidenemalonate-polysiloxane (Parsol® SLX)
tris(2-ethylhexyl)4,4′,4″-(1,3,5-triazine-2,4,6-triyltriimino)tribenzoate (Uvinul® T150)
2-ethylhexyl 2-cyano-3,3-diphenylacrylate (Neo Heliopan® 303)

Broadband filters such as, for example:
2-hydroxy-4-methoxybenzophenone-5-sulfonic acid (sulisobenzone, benzophenone-4)
or its salts.
2-hydroxy-4-methoxybenzophenone (Neo Heliopan® BB, Oxybenzone, benzophenone-3
disodium 2,2′-dihydroxy-4,4′-dimethoxy-5,5′-disulphobenzophenone
phenol,-(2H-benzotriazol-2-yl-4-methyl-6-(2-methyl-3-(1,3,3,3-tetramethyl-1-(trimethylsilyl)oxy)disiloxyanyl)propyl), (Mexoryl® XL)
2,2′-methylenebis(6-(2H-benztriazol-2-yl)-4-1,1,3,3-tetramethyl butyl)-phenol), Tinosorb® M)
2,4-bis[4-(2-ethylhexyloxy)-2-hydroxyphenyl]-1,3,5-triazine
2,4-bis[[(4-(2-ethyl hexyloxy)-2-hydroxy]phenyl]-6-(4-methoxyphenyl)-1,3,5-triazine, (Neo Heliopan® BMT)
Tris-Biphenyl Triazine (Tinosorb® A2B)
2,4-bis[[(4-(3-sulphonato)-2-hydroxypropyloxy)-2-hydroxy]phenyl]-6-(4-methoxyphenyl)-1,3,5-triazine sodium salt
2,4-bis[[(3-(2-propyloxy)-2-hydroxypropyloxy)-2-hydroxy]phenyl]-6-(4-methoxyphenyl)-1,3,5-triazine
2,4-bis[[4-(2-ethylhexyloxy)-2-hydroxy]phenyl]-6-[4-(2-methoxyethyl-carbonyl)phenylamino]-1,3,5-triazine
2,4-bis[[4-(3-(2-propyloxy)-2-hydroxypropyloxy)-2-hydroxy]phenyl]-6-[4-(2-ethyl carboxyl)phenylamino]-1,3,5-triazine
2,4-bis[[4-(2-ethylhexyloxy)-2-hydroxy]phenyl]-6-(1-methylpyrrol-2-yl)-1,3,5-triazine
2,4-bis[[4-tris(trimethylsiloxysilylpropyloxy)-2-hydroxy]phenyl]-6-(4-methoxy phenyl)-1,3,5-triazine
2,4-bis[[4-(2″-methyl propenyloxy)-2-hydroxy]phenyl]-6-(4-methoxyphenyl)-1,3,5-triazine
2,4-bis[[4-(1′,1′,1′,3′,5′,5′,5′-heptamethylsiloxy-2″-methylpropyloxy)-2 hydroxy]phenyl]-6-(4-methoxyphenyl)-1,3,5-triazine.
(5,6,5′,6′-teraphenyl-3,3′-(1,4-Phenylene)bis(1,2,4-triazine),

UVA filters are for example the following:
terephthalylidenedibornanesulphonic acid and salts (Mexoryl® SX)
Avobenzone (Neo Heliopan® 357)
hexyl 2-(4-diethylamino-2-hydroxybenzoyl)benzoate (Uvinul® A Plus)
menthyl anthranilate (Neo Heliopan® MA)

In a preferred embodiment according to the invention the composition further comprises at least one UV filter selected from the group consisting of Octisalate, Homosalate, Avobenzone or Octocrylene.

It is possible, furthermore, to use particulate UV filters or inorganic pigments, which if desired may have been rendered hydrophobic, such as the oxides of zinc (ZnO), of oxides of titanium (TiO2) of iron (Fe2O3), of zirconium (ZrO2), of silicon (SiO2), of manganese (e.g. MnO), of aluminium (Al2O3), of cerium (e.g. Ce2O3) and/or mixtures. In a further preferred embodiment according to the invention the composition further comprises one or more of Zinc Oxide, Titanium Dioxide, Octinoxate or Ensulizole.

In one embodiment according to the invention the total amount of oil soluble UV filters that can be used, which are, for example but not limited to avobenzone, and/or 2-ethylhexyl 4-dimethylaminobenzoate, and/or meradimate, and/or 2-ethylhexyl salicylate, and/or homosalate, and/or octinoxate, and/or octocrylene, is in the range of 0.1 to 55% by weight, particularly in the range of 0.5 to 40% by weight, most particularly in the range of 1 to 30% by weight, based on the total weight of the composition.

In one embodiment according to the invention the amount of octinoxate is in the range of 0.1 to 20.0% by weight, preferably in the range from 0.3 to 15% by weight and most preferably in the range from 0.5 to 10.0% by weight, based on the total weight of the composition.

In one embodiment according to the invention the amount of octocrylene is in the range of 0.1 to 20.0% by weight, preferably in the range from 0.3 to 15% by weight and most preferably in the range from 0.5 to 10.0% by weight, based on the total weight of the composition.

In one embodiment according to the invention the amount of salicylate esters is in the range of 0.1 to 20.0% by weight, preferably in the range from 0.3 to 15% by weight and most preferably in the range from 0.5 to 10.0% by weight, based on the total weight of the composition.

When Octisalate is chosen as the UV filter, it is advantageous that its total amount ranges from 0.1 to 5.0% by weight, based on the total weight of the composition. When Homosalate is chosen as the UV filter it is advantageous that its total amount ranges from 0.1 to 15.0% by weight, based on the total weight of the composition.

In one embodiment according to the invention the amount of Avobenzone is in the range of 0.1 to 10.0% by weight, preferably in the range from 0.3 to 7.0% by weight and most preferably in the range from 0.5 to 5.0% by weight, based on the total weight of the composition.

In one embodiment according to the invention the amount of Ensulizole is in the range of 0.1 to 10.0% by weight, preferably in the range from 0.3 to 8.0% by weight and most preferably in the range from 0.5 to 5.0% by weight, based on the total weight of the composition.

In one embodiment according to the invention the amount of Bemotrizinol is in the range of 0.1 to 10.0% by weight, preferably in the range from 0.3 to 7.0% by weight and most preferably in the range from 0.5 to 5.0% by weight, based on the total weight of the composition.

The total amount of micro fine organic and/or inorganic pigments, for example but not limited to Zinc Oxide (coated and un-coated), and/or titanium dioxide (coated or un-coated) that may be used in compositions according to the invention can be in the range of 0.1 to 35% by weight, preferably in the range from 0.3 to 25% by weight and more preferably in the range from 0.5 to 20.0% by weight and most preferably in the range from 0.75% to 10.0% by weight, based on the total weight of the composition. When titanium dioxide is chosen as the UV filter, it is advantageous that its total amount ranges from 0.1% to 20.0% by weight, based on the total weight of the composition. When Zinc Oxide is chosen as the UV filter, it is advantageous that its total amount ranges from 0.1% to 20.0% by weight, based on the total weight of the composition.

Synergies of 2,6-diethylhexyl naphthalate together with other constituents that do not absorb UV light, with regard to an improved protection against sunlight, are to be expected in compositions according to the invention. Non limiting examples herefore are: polymers, emulsifiers (anionic, cationic, zwitterionic, non-ionic, quaternaries), thickeners, rheology modifiers, C2- to C50 alkyl (branched or linear) esters or alkyl (branched or linear) aromatic esters, triols or their esters, glycols or their esters, 1-2 glycols, monohydric alcohols or their esters, waxes, silicone derivatives, chelating agents, preservation agents, vitamins and their derivatives, tanning agents, tanning accelerators, skin whitening or lightening agents, amino acids and their derivatives, peptides and their derivatives, carotenoids ad their derivatives, anti-inflammatory ingredients, fragrances, cooling or heating agents, insect repellents, flavonoids, anti-oxidants, plant extracts, and non-nano sized pigments (coloured or white). In a preferred embodiment according to the invention the composition further comprises at least one alkyl 1,2-diols with an alkyl chain length of 5 to 10. In a further preferred embodiment according to the invention the composition further comprises Hydroxyacetophenone.

The composition according to the invention can be in the use forms conventionally used, i.e. in the form of oil-in-water, water-in-oil or mixed emulsion, in the form of milk, in the form of lotion or cream, aerosol, hydrodispersion gel or oil gel (emulsifier-free), spray, foam, solution, powder, pencil preparation or in the form of any other customary cosmetic, dermatological and pharmacological preparations. Preparations such as shampoo, rinse, conditioner, gel, lotion, spray or cream are preferably used for protection of the hair against UV rays. In one preferred embodiment according to the invention the composition is an oil in water emulsion. In another preferred embodiment according to the invention the composition is a water in oil emulsion. In another preferred embodiment according to the invention the composition is an alcoholic spray.

The composition according to the present invention can have the customary composition and can be used for cosmetic and/or dermatological sun protection. Accordingly, the compositions according to the present invention can, depending on their formulation, be used, for example, as skin protection cream, facial moisturizer, sunscreen lotion, nourishing cream, day cream or night cream. Typical embodiments are creams, gels e.g. but not limited to hydrogels, hydrodispersion gels, oil gels; lotions, alcoholic and aqueous/alcoholic solutions, emulsions in their various forms for example but not limited to oil in water (O/W), water in oil (W/O), mixed emulsions, PIT emulsions, Pickering emulsions, microemulsions, nano-emulsions; aerosol foams, non-aerosol foams, aerosol sprays, non-aerosol sprays, pump sprays, serums, roll-ons, pastes, balsams, or stick preparations. These compositions may also comprise, as further auxiliaries and additives, mild surfactants, co-emulsifiers, super fatting agents, pearlescent waxes, bodying agents, thickeners, polymers, silicone compounds, fats, waxes, stabilizers, biogenic active ingredients, deodorant active ingredients, antidandruff agents, film formers, swelling agents, hydrotropic agents, preservatives, insect repellants, tanning agents, artificial self-tanning agents (e.g. dihydroxyacetone), stabilizers, perfume oils, dyes, antimicrobial agents, aqueous and non-aqueous plant extracts and the like. The amounts of cosmetic or dermatological auxiliaries and carrier substances and perfume which can be used in each case can be determined easily by the person skilled in the art by simple trial and error, depending on the nature of the product in question. In a preferred embodiment according to the invention the composition is a suncare formulation with an SPF of at least 30.

The cosmetic, dermatological and pharmacological compositions according to the present invention are applied to the skin and/or the hair in a sufficient amount in the manner customary for cosmetics or pharmacological and dermatological compositions.

Preferred embodiments of the cosmetic and/or pharmaceutical, especially dermatological compositions of the invention may also comprise anionic, cationic, nonionic and/or amphoteric surfactants (included in the term surfactant is the term emulsifier). Surfactants are amphiphilic substances which can dissolve or disperse organic, nonpolar substances in water. In this context, the hydrophilic components of a surfactant molecule are usually polar functional groups, for example —COO⁻, —OSO₃²⁻, —SO₃⁻, while the hydrophobic parts as a rule are nonpolar hydrocarbon radicals. Surfactants are in general classified according to the nature and charge of the hydrophilic molecular moiety. A distinction can be made between four groups here:
anionic surfactants,
cationic surfactants,
amphoteric surfactants and
nonionic surfactants.

Anionic surfactants as a rule contain carboxylate, sulphate or sulphonate groups as functional groups. In aqueous solution, they form negatively charged organic ions in an acid or neutral medium. Cationic surfactants are almost exclusively characterized by the presence of a quaternary ammonium group. In aqueous solution, they form positively charged organic ions in an acid or neutral medium. Amphoteric surfactants contain both anionic and cationic groups and accordingly behave like anionic or cationic surfactants in aqueous solution, depending on the pH. In a strongly acid medium they have a positive charge, and in an alkaline medium a negative charge. On the other hand, they are zwitterionic in the neutral pH range. Polyether and polysaccharide chains are typical of nonionic surfactants. Nonionic surfactants do not form ions in an aqueous medium.

A. Anionic Surfactants

Anionic surfactants which are advantageously used are acylamino acids (and salts thereof), such as:
acyl glutamates, for example sodium acyl glutamate, di-TEA-palmitoyl aspartate and sodium caprylic/capric glutamate,
acyl peptides, for example palmitoyl hydrolysed milk protein, sodium cocoyl hydrolysed soya protein and sodium/potassium cocoyl hydrolysed collagen,
sarcosinates, for example myristoyl sarcosine, TEA-lauroyl sarcosinate, sodium lauroyl sarcosinate and sodium cocoyl sarcosinate,
taurates, for example sodium lauroyl taurate and sodium methylcocoyl taurate, acyl lactylates, lauroyl lactylate, caproyl lactylate
alaninates
carboxylic acids and derivatives, such as for example: TEA stearate, Glyceryl stearates, PEG glyceryl stearates, lauric acid, aluminium stearate, magnesium alkanolate and zinc undecylenate,
ester-carboxylic acids, for example: calcium stearoyl lactylate, laureth-6 citrate and sodium PEG-4 lauramide carboxylate, glyceryl stearates, glyceryl-oleylstearates, glyceryl citrates, glyceryl oleyl citrates,
ether-carboxylic acids, for example sodium laureth-13 carboxylate and sodium PEG-6 cocamide carboxylate,
Glucoside esters, such as for example: cetearyl glucoside, lauryl glucoside
phosphoric acid esters and salts, such as, for example: cetyl phosphate (mono, di cetyl and their mixtures), Potassium cetyl phosphate, (mono, di cetyl and their mixtures), DEA cetyl phosphate (mono, di cetyl and their mixtures), DEA-oleth-10 phosphate and dilaureth-4 phosphate,
sulphonic acids and salts, such as for example: acyl isethionates, e.g. sodium/ammonium cocoyl isethionate, alkylarylsulphonates,
alkylsulphonates, for example sodium coco-monoglyceride sulphate, sodium C12-14 olefinsulphonate, sodium lauryl sulphoacetate and magnesium PEG-3 cocamide sulphate,
sulphosuccinates, for example dioctyl sodium sulphosuccinate, disodium laureth-sulphosuccinate, disodium laurylsulphosuccinate and disodium undecylenamido-MEA-sulphosuccinate
   and
sulphuric acid esters, such as: alkyl ether sulphate, for example sodium, ammonium, magnesium, MIPA, TIPA laureth sulphate, sodium myreth sulphate and sodium C12-13 pareth sulphate; alkyl sulphates, for example sodium, ammonium and TEA lauryl sulphate.

B. Cationic Surfactants

Cationic surfactants which are advantageously used are
alkylamines,
alkylimidazoles,
ethoxylated amines,
quaternary surfactants,
RNH₂CH₂CH₂COO⁻ (at pH=7)
RNHCH₂CH₂COO—B⁺ (at pH=12) B+=any desired cation, e.g. Na⁺ and
ester quats.

Quaternary surfactants contain at least one N atom which is covalently bonded to 4 alkyl or aryl groups. This leads to a positive charge, independently of the pH. Alkylbetaine, alkylamidopropylbetaine and alkylamidopropylhydroxysulphaine are advantageous. The cationic surfactants used can further preferably be chosen from the group consisting of quaternary ammonium compounds, in particular benzyltrialkylammonium chlorides or bromides, such as, for example, benzyldimethylstearylammonium chloride, and also alkyltrialkylammonium salts, for example cetyltrimethylammonium chloride or bromide, alkyldimethylhydroxyethylammonium chlorides or bromides, dialkyldimethylammonium chlorides or bromides, alkylamideethyltrimethylammonium ether sulphates, alkylpyridinium salts, for example lauryl- or cetylpyridinium chloride, imidazoline derivatives and compounds having a cationic character, such as amine oxides, for example alkyldimethylamine oxides or alkylaminoethyldimethylamine oxides. Cetyltrimethyl-ammonium salts in particular are advantageously used.

C. Amphoteric surfactants

Amphoteric surfactants which are advantageously to be used are
acyl/dialkylethylenediamine, for example sodium acylamphoacetate, disodium acylamphodipropionate, disodium alkylamphodiacetate, sodium acylamphohydroxypropylsulphonate, disodium acylamphodiacetate and sodium acylamphopropionate,
N-alkylamino acids, for example aminopropyl alkylglutamide, alkylaminopropionic acid, sodium alkylimidodipropionate and lauroamphocarboxyglycinate.
acylamphohydroxypropylsulphonate, disodium acylamphodiacetate and sodium acylamphopropionate,
N-alkylamino acids, for example aminopropyl alkylglutamide, alkylaminopropionic acid, sodium alkylimidodipropionate and lauroamphocarboxyglycinate.

D. Nonionic Surfactants

Nonionic surfactants which are advantageously used are
alcohols,
alkanolamides, such as cocamides MEA/DEA/MIPA,
amine oxides, such as cocoamidopropylamine oxide,
ethers, for example ethoxylated/propoxylated alcohols, ethoxylated/propoxylated esters, ethoxylated/propoxylated glycerol esters, ethoxylated/propoxylated cholesterols, ethoxylated/propoxylated triglyceride esters, ethoxylated/propoxylated lanolin, ethoxylated/propoxylated polysiloxanes, propoxylated POE ethers and alkyl polyglycosides, such as lauryl glucoside, decyl glucoside and coco-glycoside.
sucrose esters, sucrose ethers
polyglycerol esters, diglycerol esters, monoglycerol esters polyglyceryl-2 dipolyhydroxystearate (Dehymuls® PGPH), polyglyceryl-3 diisostearate (Lameform® TGI), polyglyceryl-4 isostearate (Isolan® GI 34), polyglyceryl-3 oleate, diisostearyl polyglyceryl-3 diisostearate (Isolan® PDI), polyglyceryl-3 methylglucose distearate (Tego Care® 450), polyglyceryl-3 beeswax (Cera Bellina®), polyglyceryl-4 caprate (polyglycerol caprate T2010/90), polyglyceryl-3 cetyl ether (Chimexane® NL), polyglyceryl-3 distearate (Cremophor® GS 32), polyglyceryl-2 stearate (Hostacerin® DGMS) and polyglyceryl polyricineoleate (Admul® WOL 1403), and mixtures thereof.
methylglucose esters, esters of hydroxy acids

The use of a combination of anionic and/or amphoteric surfactants with one or more nonionic surfactants is further advantageous. In a preferred embodiment according to the invention the composition further comprises emulsifiers selected from the group consisting of:
a) Alkyl phosphate derivatives
b) Gylceryl oleate citrate derivatives
c) Glycereyl stearate citrate derivatives
d) Stearic acid esters
e) Sorbitan esters
f) Ethoxylated sorbitan esters
g) Ethoxylated mono-, di- and tri glycerides
h) Methyl glucose esters

In addition, compositions according to the present invention can advantageously, but not obligatorily, comprise inorganic pigments based on finely disperse metal oxides and/or other metal compounds which are insoluble or sparingly soluble in water, in particular the oxides of titanium (TiO₂), zinc (ZnO), iron (e.g. Fe₂O₃), zirconium (ZrO₂), silicon (SiO₂), manganese (e.g. MnO), aluminum (Al₂O₃), cerium (e.g. Ce₂O₃), mixed oxides of the corresponding metals, and mixtures of such oxides. These pigments are X-ray-amorphous or non-X-ray-amorphous. X-ray-amorphous oxide pigments are metal oxides or semi-metal oxides which reveal no or no recognizable crystalline structure in X-ray diffraction experiments. Such pigments are often obtainable by flame reaction, for example by reacting a metal or semi-metal halide with hydrogen and air (or pure oxygen) in a flame.

X-ray-amorphous oxide pigments are used as thickeners and thixotropic agents, flow auxiliaries for emulsion and dispersion stabilization and as carrier substance (for example for increasing the volume of finely divided powders). X-ray-amorphous oxide pigments which are known and often used in cosmetic or dermatological galenics are, for example, high-purity silicon oxide. Preference is given to high-purity, X-ray-amorphous silicon dioxide pigments with a particle size in the range from 5 to 40 nm and an active surface area (BET) in the range from 50 to 400 m²/g, preferably 150 to 300 m²/g, where the particles are to be regarded as spherical particles of very uniform dimension. Macroscopically, the silicon dioxide pigments are recognizable as loose, white powders. Silicon dioxide pigments are sold commercially under the name Aerosil® (CAS-No. 7631-85-9) or Carb-O-Sil

Advantageous Aerosil grades are, for example, Aerosil® 0X50, Aerosil® 130, Aerosil® 150, Aerosil® 200, Aerosil® 300, Aerosil® 380, Aerosil® MQX 80, Aerosil® MOX 170, Aerosil® COK 84, Aerosil® R 202, Aerosil® R 805, Aerosil® R 812, Aerosil® R 972, Aerosil® R 974, Aerosil® R976.

The compositions according to the present invention can comprise 0.1 to 20% by weight, advantageously 0.5 to 10% by weight, more preferably 1 to 5% by weight, based on the total weight of the compositions, of X-ray-amorphous oxide pigments.

The non-X-ray-amorphous inorganic pigments are, according to the present invention, advantageously in hydrophobic form, i.e. have been surface-treated to repel water. This surface treatment may involve providing the pigments with a thin hydrophobic layer by processes known per se. Such a process involves, for example, producing the hydrophobic surface layer by a reaction according to

nTiO₂+m(RO)₃Si—R′→nTiO₂(surf.)

where n and m are stoichiometric parameters to be used as desired, and R and R′ are the desired organic radicals. Hydrophobic pigments prepared analogously to DE-A 33 14 742, for example, are advantageous.

The total amount of inorganic pigments, in particular hydrophobic inorganic micro pigments, in the finished cosmetic, dermatological and pharmacological composition according to the invention can be advantageously chosen from the range from 0.1 to 30% by weight, preferably 0.1 to 10.0% by weight, preferably 0.5 to 6.0% by weight, based on the total weight of the compositions.

An additional content of skin lightening ingredients in the compositions according to the present invention is optional. Such skin lightening ingredients which can be used are for example but not limited to the following: kojic acid (5-hydroxy-2-hydroxymethyl-4-pyranone), kojic acid derivatives such as for example kojic dipalmitate, arbutin, ascorbic acid, ascorbic acid derivatives, hydroquinone, hydroquinone derivatives, styryl resorcinol derivatives (e.g. 4-(1-phenylethyl)1,3-benzenediol), molecules containing sulphur, such as glutathione or cysteine for example, alpha-hydroxy acids (e.g. citric acid, lactic acid, malic acid) and their derivatives, N-acetyltyrosine and derivatives, undecenoylphenylalanine, gluconic acid, chromone derivatives such as aloesin, flavonoids, thymol derivatives, 1-aminoethylphosphinic acid, thiourea derivatives, ellagic acid, nicotinamide, zinc salts such as zinc chloride or zinc gluconate for example, thujaplicin and derivatives, triterpenes such as maslic acid, sterols such as ergosterol, benzofuranones such as senkyunolide, vinyl- and ethylguaiacol, dionic acids such as octodecenedionic acid and azelaic acid, nitrogen oxide synthesis inhibitors such as L-nitroarginine and its derivatives, 2,7-dinitroindazole or thiocitrulline, metal chelators (e.g. alpha-hydroxy fatty acids, palmitic acid, phytic acid, lactoferrin, humic acid, gallic acid, bile extracts, bilirubin, biliverdin), retinoids, soja milk, soya extract, serine protease inhibitors or lipoic acid or other synthetic or natural active compounds for skin and hair lightening, these compounds also being used in the form of an extract from plants, such as bearberry extract, rice extract, papaya extract, liquorice root extract or constituents concentrated from these, such as glabridin or licochalcone A, Artocarpus extract, extract from Rumex and Ramulus species, extracts from pine species (Pinus) and extracts from Vitis species or stilbene derivatives concentrated from these, extract from saxifraga, mulberry, Scutelleria and/or grapes.

An additional content of antioxidants in the compositions of the present invention is generally preferred. According to the present invention, favorable antioxidants which can be used are all antioxidants customary or suitable for cosmetic, dermatological and pharmacological preparations. The antioxidants are advantageously chosen from the group of amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. α-carotene, β-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g. buthionine sulfoximines, homocysteine sulfoximine, buthionine sulfones, penta-, hexa-, heptathionine sulfoximine) in very low tolerated doses (e.g. pmol to μmol/kg), and also (metal) chelating agents (e.g. α-hydroxy fatty acids, palmitic acid, phytic acid, lactoferrin), α-hydroxy acids (e.g. citric acid, lactic acid, maleic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate), and coniferyl benzoate of benzoin resin, rutinic acid and derivatives thereof, α-glycosylrutin, ferulic acid, furfurylideneglucitol, carnosine, butylhydroxy-toluene, butylhydroxyanisol, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids), derivatives of acetophenone such as Hydroxyacetophenone and its blends with Phenoxyethanol and/or, pentane 1,2 diol and/or hexane 1,2 diol and/or caprylyl 1,2 diol, are suitable according to the present invention.

The amount of the above-mentioned antioxidants (one or more compounds) in the composition is preferably 0.001 to 30% by weight, more preferably 0.05 to 20% by weight, and most preferably 1 to 10% by weight, based on the total weight of the composition.

In a preferred embodiment the composition of the invention may advantageously also comprise vitamins and vitamin precursors, it being possible for all the vitamins and vitamin precursors which are suitable or usual for cosmetic and/or dermatological applications to be used. Those worth mentioning here are, in particular, vitamins and vitamin precursors, such as tocopherols, vitamin A, niacin acid and niacinamide, further vitamins of the B complex, in particular biotin, and vitamin C and panthenol and derivatives thereof, in particular the esters and ethers of panthenol, and cationically derivatized panthenols, such as panthenol triacetate, panthenol monoethyl ether and the monoacetate thereof and cationic panthenol derivatives. If vitamin E and/or derivatives thereof represent the antioxidant(s), it is advantageous to choose their respective concentrations from the range from 0.001 to 10% by weight, based on the total weight of the composition. If vitamin A or vitamin A derivatives, or carotenes or derivatives thereof represent the antioxidant(s), it is advantageous to choose their respective concentrations from the range from 0.001 to 10% by weight, based on the total weight of the composition.

In a preferred embodiment of the composition of the invention may also comprise lipids chosen from the following group of substances:

(i) linear or branched saturated paraffins (mineral oils) having 15 or more C atoms, in particular having 18 to 45 C atoms;
(ii) esters having 12 or more C atoms of linear or branched fatty acids having 6 to 30 C atoms and linear or branched, saturated or unsaturated mono-, di- or triols having 3 to 30 C atoms, these esters having no free hydroxyl groups;
(iii) esters of benzoic acid and linear or branched, saturated or unsaturated monoalkanols having 8 to 20 C atoms;
(iv) monoesters or diesters of alcohols having 3 to 30 C atoms and naphthalene-monocarboxylic or -dicarboxylic acids; especially naphthalenemonocarboxylic acid C₆-C₁₈ esters and naphthalenedicarboxylic acid di-C₆-C₁₈ esters;
(v) linear or branched, saturated or unsaturated di-C₆-C₁₈-alkyl ethers;
(vi) silicone oils;
(vii) 2-alkyl-1-alkanols of the formula (III)

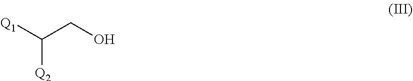

where
Q₁ is a linear or branched alkyl radical having 6 to 24 C atoms and
Q₂ is a linear or branched alkyl radical having 4 to 16 C atoms.

An oil phase or oil component in the narrower (and preferred) sense of the present invention, i.e. of the inventively limited substances or substances present only in a minor fraction, encompasses the following groups of substances:

(i) linear or branched, saturated paraffins having 20 to 32 C atoms;
(ii) esters having at least 14 C atoms of linear or branched, saturated fatty acids having 8 to 24 C atoms and linear or branched, saturated or unsaturated mono-, di- or triols having 3 to 24 C atoms, these esters containing no free hydroxyl groups;
(iii) esters of benzoic acid and linear or branched, saturated monoalkanols having 10 to 18 C atoms;
(iv) Alkylenediol dicaprylate caprates especially propylenediol dicapylate caprate;
(v) linear or branched, saturated di-C6-C18-alkyl ethers, especially (straight-chain) di-C6-C12-alkyl ethers;
(vi) silicone oils from the group of the cyclotrisiloxanes, cyclopentasiloxanes, dimethylpolysiloxanes, diethylpolysiloxanes, methylphenylpolysiloxanes, diphenylpolysiloxanes and hybrid forms thereof;
(vii) 2-alkyl-1-alkanols having 12 to 32 C atoms of the formula (III)
where
Q₁ is a (preferably linear) alkyl radical having 6 to 18 C atoms and
Q₂ is a (preferably linear) alkyl radical having 4 to 16 C atoms.
An oil phase in the narrowest (and most preferred) sense of the present invention encompasses the following groups of substances:
(i) linear or branched, saturated paraffins having 20 to 32 C atoms such as isoeicosane or squalane;
(ii) esters having at least 16 C atoms of linear or branched, saturated fatty acids having 8 to 18 C atoms and linear or branched, saturated mono-, di- or triols having 3 to 18 C atoms, these esters containing no free hydroxyl groups;
(iii) esters of benzoic acid and linear or branched, saturated monoalkanols having 12 to 15 C atoms, especially C₁₂₋₁₅-alkyl benzoates;
(iv) Alkylenediol dicaprylate caprates especially propylenediol dicapylate caprate;
(v) straight-chain di-C₆-C₁₀-alkyl ethers; especially di-n-octyl ether (dicaprylyl ether);
(vi) silicone oils from the group undecamethylcyclotrisiloxane, cyclomethicone, decamethylcyclopentasiloxane, dimethylpolysiloxanes, diethylpolysiloxanes, methylphenylpolysiloxanes and diphenylpolysiloxanes;
(vii) 2-alkyl-1-alkanols having 12 to 32 C atoms of the formula (III) where
Q₁ is a (preferably linear) alkyl radical having 6 to 18 C atoms and
Q₂ is a (preferably linear) alkyl radical having 4 to 16 C atoms.

Particularly preferred components of type (i) in the oil phase are as follows: isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, 2-ethylhexyl isostearate, isotridecyl isononanoate, 2-ethylhexyl cocoate, caprylic/capric triglyceride, Alkylenediol dicaprylate caprates especially propylenediol dicapylate caprate; and also synthetic, semisynthetic and natural mixtures of such esters, e.g. jojoba oil.

Fatty acid triglycerides (oil components of type (i) in the oil phase) may also be in the form of, or in the form of a constituent of, synthetic, semisynthetic and/or natural oils, examples being olive oil, sunflower oil, soya oil, peanut oil, rapeseed oil, almond oil, palm oil, coconut oil, palm kernel oil and mixtures thereof.

Particularly preferred oil components of type (vii) in the oil phase are as follows: 2-butyl-1-octanol, 2-hexyl-1-decanol, 2-octyl-1-dodecanol, 2-decyltetradecanol, 2-dodecyl-1-hexadecanol and 2-tetradecyl-1-octadecanol.

Particularly preferred oil components in the oil phase are mixtures comprising C₁₂-C₁₅-alkyl benzoate and 2-ethylhexyl isostearate, mixtures comprising C₁₂-C₁₅-alkyl benzoate and isotridecyl isononanoate, mixtures comprising C₁₂-C₁₅-alkyl benzoate, 2-ethylhexyl isostearate and isotridecyl isononanoate, mixtures comprising cyclomethicone and isotridecyl isononanoate, and mixtures comprising cyclomethicone and 2-ethylhexyl isostearate.

In preferred embodiments the composition, especially dermatologically active composition, of the invention may advantageously also comprise the use of polymers to improve the spreadibility of the composition upon the skin or hair, or improve the water and or sweat and or rub-off resistancy of the formula and to improve the protection factor of the composition. Examples of such polymers are: VP/Eicosene copolymers sold under the trade name of Antaron V-220 by International Specialty Products, VP/Hexadecene copolymer sold under the trade names Antaron V-216 and Antaron V-516 by International Specialty Products, Tricontanyl PVP sold under the trade name of Antaron WP-660 by International Specialty Products, Isohexadecane and Ethylene/Propylene/Styrene copolymer and Butylene/Styrene copolymer sold under the trade names of Versagel MC and MD by Penreco, Hydrogenated polyisobutene and Ethylene/Propylene/Styrene copolymer and Butylene/Styrene copolymer sold under the trade mane of Versagel ME by Penreco, Acrylates/Octylacrylamide Coploymers sold under the trade name of Dermacryl 79, Dermacryl AQF and Dermacryl LT by AkzoNobel, Polyurethanes such as PPG-17/IPDI/DMPA copolymer sold under the trade name of Avalure UR 450 & 525 sold by Noveon, Polyurethanes-2 and -4 sold under the trade names Avalure UR-405, -410, -425, -430 and -445 525 sold by Noveon, Polyurethane 5 and Butyl Acetate and isopropyl alcohol sold under the trade name Avalure UR-510 and -525 sold by Noveon, Polyurethanes-1 and -6 sold under the trade name of Luviset PUR by BASF, Hydrogenated Dimer Dilinoleyl/Dimethylcarbonate Copolymer sold under the trade name of Cosmedia DC by Cognis.

Of course, as one well versed in the art of cosmetic, dermatological and pharmacological compositions knows, this is not an exhaustive list and other suitable polymers not listed here may be used. Examples of such polymers may be found in the latest edition of the CTFA's International Cosmetic Ingredient Dictionary

The amount of polymers used to obtain the desired effect in the formulation range from 0.10% to 5.0% by weight of the composition and especially in the range from 0.25% to 3.0% by weight of the composition.

In preferred embodiments the composition according to the invention, especially the dermatologically active composition of the invention comprise, if desired, further ingredients having care properties, such as, for example, fatty alcohols having 6 to 30 C atoms. The fatty alcohols here can be saturated or unsaturated and linear or branched. Furthermore, these fatty alcohols can in some cases be part of the oil phase (III) if they correspond to the definition given there. Alcohols which can be employed are, for example, decanol, decenol, octanol, octenol, dodecanol, dodecenol, octadienol, decadienol, dodecadienol, oleyl alcohol, ricinoleyl alcohol, erucyl alcohol, stearyl alcohol, isostearyl alcohol, cetyl alcohol, lauryl alcohol, myristyl alcohol, arachidyl alcohol, caprylyl alcohol, capryl alcohol, linoleyl alcohol, linolenyl alcohol and behenyl alcohol, and also Guerbet alcohols thereof, such as, for example, 2-octyl-1-dodecanol, it being possible for the list to be extended virtually as desired by further alcohols of related structural chemistry. The fatty alcohols preferably originate from natural fatty acids, being conventionally prepared from the corresponding esters of the fatty acids by reduction. Fatty alcohol fractions which are formed by reduction from naturally occurring fats and fatty oils, such as beef tallow, peanut oil, colza oil, cottonseed oil, soya oil, sunflower oil, palm kernel oil, linseed oil, maize oil, castor oil, rapeseed oil, sesame oil, cacao butter and coconut fat, can further be employed.

Substances having care properties which advantageously can be employed in the cosmetic, dermatological and pharmacological compositions according to the invention can further include
ceramides, where ceramides are understood as meaning N-acylsphingosins (fatty acid amides of sphingosin) or synthetic analogues of such lipids (so-called pseudo-ceramides), which significantly improve the water retention capacity of the stratum corneum.
phospholipids, for example soya lecithin, egg lecithin and cephalins
fatty acids
phytosterols and phytosterol-containing fats or waxes
vaseline, paraffin oils and silicone oils; the latter include, inter alia, dialkyl- and alkylarylsiloxanes, such as dimethylpolysiloxane and methylphenylpolysiloxane, and also alkoxylated and quaternised derivatives thereof.

Animal and/or plant protein hydrolysates can advantageously also be added to preferred embodiments of cosmetic and/or pharmaceutical, especially dermatologically active, compositions of the invention. Substances which are advantageous in this respect are, in particular, elastin, collagen, keratin, milk protein, soya protein, oat protein, pea protein, almond protein and wheat protein fractions or corresponding protein hydrolysates, and also condensation products thereof with fatty acids, and quaternised protein hydrolysates, the use of plant protein hydrolysates being preferred.

In a preferred embodiment according to the invention the composition is an oil in water or an water in oil emulsion and the oil phase of the emulsion comprises one or more of

a) hydrocarbon oils,
b) waxes
c) silicone oils
d) natural oils
e) fatty acid esters
f) fatty alcohols
g) antioxidants
i) chelating agents
j) skin lightening agents
k) tan accelerating agents
l) insect repelling agents
m) moisturizing agents
n) water resistant polymers

It may be advantageously according to the present invention that the aqueous phase of the emulsion according to the invention comprises alcohols, diols or polyols (lower alkyl), and ethers thereof, preferably ethanol, isopropanol, propylene glycol, 1,2-pentane diol, 1,2-hexanediol, 1,2-octanediol, 1,2-decanediol, a mixture of 1,2-hexanediol and 1,2-octanediol, a mixture of 1,2-hexanediol and 1,2-decanediol, a mixture of 1,2-octanediol and 1,2-decanediol, a mixture of 1,2-hexanediol, 1,2-octanediol and 1,2-decanediol, glycerol, ethylene glycol-monoethyl or monobutyl ether, propylene glycol monomethyl, -monoethyl or monobutyl ether, diethylene glycol monomethyl or -monoethyl ether and analogous products, and also alcohols (lower alkyl), e.g. ethanol, 1,2-propanediol, glycerol, and, in particular, one or more thickeners which can advantageously be chosen from the group of silicon dioxide, aluminum silicates, polysaccharides and derivatives thereof, e.g. hyaluronic acid, xanthan gum, hydroxypropylmethylcellulose, particularly advantageously from the group of polyacrylates, preferably a polyacrylate from the group of so-called Carbomers, for example but not limited to, Carbopol® grades 980, 981, 1382, 2984, 5984, in each case individually or in combination.

In preferred embodiments according to the invention the compositions may also comprise active anti-inflammatory and/or redness- and/or itching-alleviating compounds (anti-irritants). All the active anti-inflammatory or redness- and/or itching-alleviating compounds which are suitable or usual for cosmetic, dermatological and pharmacological compositions can be used here. Active anti-inflammatory and redness- and/or itching-alleviating compounds which are advantageously employed are steroidal anti-inflammatory substances of the corticosteroid type, such as hydrocortisone, dexamethasone, dexamethasone phosphate, methylprednisolone or cortisone, it being possible for the list to be extended by addition of further steroidal anti-inflammatories. Non-steroidal anti-inflammatories can also be employed. Those to be mentioned here by way of example are oxicams, such as piroxicam or tenoxicam; salicylates, such as aspirin, Disalcid, Solprin or fendosal; acetic acid derivatives, such as diclofenac, fenclofenac, indomethacin, sulindac, tolmetin, or clindanac; fenamates, such as mefenamic, meclofenamic, flufenamic or niflumic; propionic acid derivatives, such as ibuprofen, naproxen, benoxaprofen or pyrazoles, such as phenylbutazone, oxyphenylbutazone, febrazone or azapropazone.

Alternatively, natural anti-inflammatory or redness- and/or itching-alleviating substances can be employed. Plant extracts, specific highly active plant extract fractions and highly pure active substances isolated from plant extracts can be employed. Extracts, fractions and active substances from chamomile, aloe vera, Commiphora species, Rubia species, willow, rose-bay willow-herb, oats, and also pure substances, such as, inter alia, bisabolol, apigenin 7-glucoside, boswellic acid, phytosterols, glycyrrhizic acid, glabridin or licochalcone A, are particularly preferred. The compositions of the present invention can also comprise mixtures of two or more active anti-inflammatory compounds. Bisabolol, boswellic acid, and also extracts and isolated highly pure active compounds from oats and Echinacea are particularly preferred for use in the context of the invention as anti-inflammatory and redness- and/or itching-alleviating substances, and alpha-bisabolol and extracts and isolated highly pure active compounds from oats are especially preferred.

The amount of anti-irritants (one or more compounds) in the composition is preferably 0.0001% to 20% by weight, with particular preference 0.0001% to 10% by weight, in particular 0.001% to 5% by weight, based on the total weight of the composition.

In preferred embodiments of the cosmetic and/or pharmaceutical, especially dermatologically active compositions of the invention may advantageously also comprise moisture retention regulators. The following substances for example are used as moisture retention regulators (moisturizers): sodium lactate, urea, alcohols, sorbitol, glycerol, propylene glycol, aliphatic 1,2-diols with a C number of 5-10, collagen, elastin or hyaluronic acid, diacyl adipates, petrolatum, ectoin, urocanic acid, lecithin, panthenol, phytantriol, lycopene, algae extract, ceramides, cholesterol, glycolipids, chitosan, chondroitin sulphate, polyamino acids and polyamino sugars, lanolin, lanolin esters, amino acids, alpha-hydroxy acids (e.g. citric acid, lactic acid, malic acid) and derivatives thereof, sugars (e.g. inositol), alpha-hydroxy fatty acids, phytosterols, triterpene acids, such as betulinic acid or ursolic acid, algae extracts.

Preferred embodiments are compositions which also comprise mono-, di- and oligosaccharides, such as, for example, glucose, galactose, fructose, mannose, fruit sugars and lactose.

Further preferred embodiments according to the invention are cosmetic and/or pharmaceutical, especially dermatologically active compositions which also comprise plant extracts, which are conventionally prepared by extraction of the whole plant, but also in individual cases exclusively from blossom and/or leaves, wood, bark or roots of the plant. In respect of the plant extracts which can be used, reference is made in particular to the extracts which are listed in the table starting on page 44 of the 3rd edition of the Leitfaden zur Inhaltsstoffdeklaration kosmetischer Mittel [Manual of Declaration of the Constituents of Cosmetic Compositions], published by Industrieverband Körperpflegemittel und Waschmittel e.V. (IKW), Frankfurt. Extracts which are advantageous in particular are those from aloe, witch hazel, algae, oak bark, rose-bay willow-herb, stinging nettle, dead nettle, hops, chamomile, yarrow, arnica, calendula, burdock root, horsetail, hawthorn, linden blossom, almond, pine needle, horse chestnut, sandalwood, juniper, coconut, mango, apricot, orange, lemon, lime, grapefruit, apple, green tea, grapefruit pip, wheat, oats, barley, sage, thyme, wild thyme, rosemary, birch, mallow, lady's smock, willow bark, restharrow, coltsfoot, hibiscus, ginseng and ginger root.

In this context, the extracts from aloe vera, chamomile, algae, rosemary, calendula, ginseng, cucumber, sage, stinging nettle, linden blossom, arnica and witch hazel are particularly preferred. Mixtures of two or more plant extracts can also be employed. Extraction agents which can be used for the preparation of plant extracts mentioned are, inter alia, water, alcohols and mixtures thereof. In this context, among the alcohols lower alcohols, such as ethanol and isopropanol, but also polyhydric alcohols, such as ethylene glycol, propylene glycol and butylene glycol, are preferred, and in particular both as the sole extraction agent and in mixtures with water. The plant extracts can be employed both in pure and in diluted form.

Compositions which are preferred embodiments of the invention may in numerous cases advantageously comprise the following preservatives:

Preservatives which are preferably chosen here are those such as benzoic acid, its esters and salts, propionic acid and its salts, salicylic acid and its salts, 2,4-hexadienoic acid (sorbic acid) and its salts, formaldehyde and paraformaldehyde, 2-hydroxybiphenyl ether and its salts, 2-zincsulphidopyridine N-oxide, inorganic sulphites and bisulphites, sodium iodate, chlorobutanolum, 4-ethylmercuryl(II)-5-amino-1,3-bis(2-hydroxybenzoic acid), its salts and esters, dehydracetic acid, formic acid, 1,6-bis(4-amidino-2-bromophenoxy)-n-hexane and its salts, the sodium salt of ethylmercury(II)-thiosalicylic acid, phenylmercury and its salts, 10-undecylenic acid and its salts, 5-amino-1,3-bis(2-ethylhexyl)-5-methyl-hexahydropyrimidine, 5-bromo-5-nitro-1,3-dioxane, 2-bromo-2-nitro-1,3-propanediol, 2,4-dichlorobenzyl alcohol, N-(4-chlorophenyl)-N′-(3,4-dichlorophenyl)urea, 4-chloro-m-cresol, 2,4,4′-trichloro-2′-hydroxydiphenyl ether, 4-chloro-3,5-dimethylphenol, 1,1′-methylene-bis(3-(1-hydroxymethyl-2,4-dioximidazolidin-5-yl)urea), poly(hexamethylene diguanide) hydrochloride, 2-phenoxyethanol, hexamethylenetetramine, 1-(3-chloroallyl)-3,5,7-triaza-1-azoniaadamantane chloride, 1-(4-chlorophenoxy)-1-(1H-imidazol-1-yl)-3,3-dimethyl-2-butanone, 1,3-bis-(hydroxymethyl)-5,5-dimethyl-2,4-imidazolidinedione, benzyl alcohol, octopirox, 1,2-dibromo-2,4-dicyanobutane, 2,2′-methylenebis(6-bromo-4-chlorophenol), bromochlorophene, mixture of 5-chloro-2-methyl-3(2H)-isothiazolinone and 2-methyl-3(2H)isothiazolinone with magnesium chloride and magnesium nitrate, 2-benzyl-4-chlorophenol, 2-chloroacetamide, chlorhexidine, chlorhexidine acetate, chlorhexidine gluconate, chlorhexidine hydrochloride, 1-phenoxypropan-2-ol, N-alkyl(C₁₂-C₂₂)trimethylammonium bromide and chloride, 4,4-dimethyl-1,3-oxazolidine, N-hydroxymethyl-N-(1,3-di(hydroxymethyl)-2,5-dioxoimidazolidin-4-yl)-N′-hydroxymethylurea, 1,6-bis(4-amidinophenoxy)-n-hexane and its salts, glutaraldehyde, 5-ethyl-1-aza-3,7-dioxabicyclo[3.3.0]octane, 3-(4-chlorophenoxy)-1,2-propanediol, hyamines, alkyl-(C₈-C₁₈)-dimethylbenzylammonium chloride, alkyl-(C₈-C₁₈)-dimethylbenzylammonium bromide, alkyl-(C₈-C₁₈)-dimethylbenzyl-ammonium saccharinate, benzyl hemiformal, 3-iodo-2-propynyl butylcarbamate, sodium hydroxymethylaminoacetate or sodium hydroxymethylaminoacetate.

In a preferred embodiment according to the invention the composition is a oil in water or a water in oil emulsion and the aqueous phase of the emulsion comprises one or more of:

a) antioxidants,
b) preservation agents
c) chelating agents

In various cases it may also be advantageous to employ substances which are chiefly employed for inhibition of the growth of undesirable microorganisms on or in animal organisms in compositions of the invention. In this respect, in addition to conventional preservatives, further active compounds which are worth mentioning, in addition to the large group of conventional antibiotics, are, in particular, the products relevant for cosmetics, such as triclosan, climbazol, octoxyglycerol, octopirox (1-hydroxy-4-methyl-6-(2,4,4-trimethylpentyl)-2(1H)-pyridone, 2-aminoethanol), chitosan, farnesol, glycerol monolaurate or combinations of the substances mentioned, which are employed, inter alia, against underarm odour, foot odour or dandruff formation. Also ingredients which have multifunctional properties including the ability to reduce the growth of bacteria, yeast and molds may be employed to compositions covered by the invention. These may include, but are not restricted to pentane 1,2-diol, hexane 1,2-diol, caprylyl 1,2-diol, decyl 1,2-diol, tropolone, hydroxyacetophenone, ethylhexyl glycerin, phenoxyethanol either as individual ingredients or a mixtures of 2 or more of these.

Furthermore, the compositions of the invention may also comprise substances having a cooling action. Individual active cooling compounds which are preferred for use in the context of the present invention are listed below. The skilled person is able to supplement the following list with a large number of further active cooling compounds; the active cooling compounds listed can also be employed in combination with one another: l-menthol, d-menthol, racemic menthol, menthone glycerol acetal (trade name: Frescolat® MGA), menthyl lactate (trade name: Frescolat® ML, menthyl lactate is preferably l-menthyl lactate, in particular l-menthyl l-lactate), menthyl ethylamido oxalate (Frescolat® X-Cool), substituted menthyl-3-carboxylic acid amides (e.g. menthyl-3-carboxylic acid N-ethylamide), 2-isopropyl-N-2,3-trimethylbutanamide, substituted cyclohexanecarboxylic acid amides, 3-menthoxypropane-1,2-diol, 2-hydroxyethyl menthyl carbonate, 2-hydroxypropyl menthyl carbonate, N-acetylglycine menthyl ester, isopulegol, menthyl hydroxycarboxylic acid esters (e.g. menthyl 3-hydroxybutyrate), monomenthyl succinate, 2-mercaptocyclodecanone, menthyl 2-pyrrolidin-5-onecarboxylate, 2,3-dihydroxy-p-menthane, 3,3,5-trimethylcyclo-hexanone glycerol ketal, 3-menthyl 3,6-di- and -trioxaalkanoates, 3-menthyl methoxyacetate, icilin.

Preferred active cooling compounds are: l-menthol, d-menthol, racemic menthol, menthone glycerol acetal (trade name: Frescolat® MGA), menthyl lactate (preferably l-menthyl lactate, in particular l-menthyl l-lactate, trade name: Frescolat® ML), menthyl ethylamido oxalate (Frescolat® X-Cool), substituted menthyl-3-carboxylic acid amides (e.g. menthyl-3-carboxylic acid N-ethylamide), 2-isopropyl-N-2,3-trimethylbutanamide, substituted cyclohexanecarboxylic acid amides, 3-menthoxypropane-1,2-diol, 2-hydroxtethyl menthyl carbonate, 2-hydroxypropyl menthyl carbonate, isopulegol.

Particularly preferred active cooling compounds are: l-menthol, racemic menthol, menthone glycerol acetal (trade name: Frescolat® MGA), menthyl lactate (preferably l-menthyl lactate, in particular l-menthyl l-lactate, trade name: Frescolat® ML), menthyl ethylamido oxalate (Frescolat® X-Cool), 3-menthoxypropane-1,2-diol, 2-hydroxyethyl menthyl carbonate, 2-hydroxypropyl menthyl carbonate.

Very particularly preferred active cooling compounds are: l-menthol, menthone glycerol acetal (trade name: Frescolat® MGA), menthyl lactate (preferably l-menthyl lactate, in particular l-menthyl l-lactate, trade name: Frescolat® ML) or menthyl ethylamido oxalate (Frescolat® X-Cool).

The use concentration of the active cooling compounds to be employed is, depending on the substance, preferably in the concentration range from 0.01% to 20% by weight, and more preferably in the concentration range from 0.1% to 5% by weight, based on the total weight of the completed (ready-to-use) cosmetic, dermatological and pharmacological composition.

In a further preferred embodiment according to the invention the composition has a UVA protection factor of at least 370 nm, as measured by the Critical Wavelength Method for in vitro determination of UVA protection. The critical wavelength is defined as the wavelength at which the integral of the spectral absorbance curve reaches 90% of the integral over the UV spectrum from 290 to 400 nm. The method of its determination is described in the Federal Register FR volume 77, No. 92, Jun. 17, 2011 pages 35664-3565. The minimum critical wavelength allowed to fulfil the broad spectrum protection claim is 370 nm.

In a further preferred embodiment according to the invention the composition is a dermatological composition, more preferred a dermatological active composition.

Furthermore, a method for obtaining the composition according to the invention is provided herein, comprising the following steps:

(a) providing at least one oil phase comprising Diethylhexyl 2,6-Naphthalate;
(b) providing at least one aqueous phase;
wherein said oil phase and said aqueous phase are free of oxybenzone;
(c) heating and homogenizing each of said oil phase and said aqueous phase separately;
(d) adding said aqueous phase to said oil phase to a mixture; and
(e) homogenizing said mixture.

In one embodiment according to the invention, the oil phase and the aqueous phase are heated before step (c) to temperatures between 70 and 90° C.

It goes without saying that all previously mentioned embodiments of the invention also apply to the method.

The following examples are intended to illustrate the present invention without restricting it. All amounts quoted, proportions and percentages are, unless indicated otherwise, based on the weight and the total amount or on the total weight of the compositions.

### EXAMPLES

Comparison of Absorbance Spectra of Benzophene-3 and Diethylhexyl 2,6-Naphthalate.

A typical suncare formulation with an SPF greater than 50 in the US market contains 9.0% by weight Octocrylene+9.0% by weight Homoslate+5.0% by weight Octisalate+2.3% by weight Avobenzone+6.0% by weight Oxybenzone. The UV absorbance curve of 10 mg/l of this combination of UV filters in solution has a maximum absorbance in the UVB range of the spectrum of about 1.0 AU falling to 0.36 AU at 370 nm (see FIG. 1 spectrum 1). Removing the Oxybenzone from this formula results, as expected in a much lower absorbance with a maximum of 0.75 AU in the UVB region 0.32 AU at 370 nm (see FIG. 1 spectrum 2). The addition of 10% Diethylhexyl Naphthalate to the mixture, as expected results only in a slight increase in the absorbance curve in the UVB region with no increase at 370 nm (see FIG. 1 spectrum 3). We would therefore expect that the formulae 2 and 3 without benzophenone 3 would have similar SPFs but with a much lower SPF than formula 1.

An emulsion (Table 1) was made with variants 1 and 3 described above.

TABLE 1

| | 1 % | 3 % |
|---|---|---|
| INCI | w/w | w/w |
| Potassium Cetyl Phosphate, Hydrogenated Palm | 2.00 | 2.00 |
| Glycerides | | |
| Butyl Methoxydibenzoylmethane | 2.70 | 2.70 |
| Octocrylene | 9.00 | 9.00 |
| Homosalate | 9.00 | 9.00 |
| Ethylhexyl Salicylate | 5.00 | 5.00 |
| Benzophenone-3 | 6.00 | 0.00 |
| Caprylic/Capric Triglyceride | 10.00 | 6.00 |
| Disodium EDTA | 0.10 | 0.10 |
| Cyclopentasiloxane, Acrylates/Dimethicone | 1.00 | 1.00 |
| Copolymer | | |
| Acrylates/C10-30 Alkyl Acrylate Crosspolymer | 0.25 | 0.25 |
| Xanthan Gum | 0.10 | 0.10 |
| Diethylhexyl 2,6-Naphthalate | 0.00 | 10.00 |
| Water (Aqua) | 50.15 | 50.15 |
| Sodium Hydroxide | 0.70 | 0.70 |
| Glycerin | 3.00 | 3.00 |
| Hydroxyacetophenone | 0.50 | 0.50 |
| 1,2-Hexanediol, Caprylyl Glycol | 0.50 | 0.50 |
| Total | 100.00 | 100.00 |

It was therefore expected that the absorbance characteristics of a thin film an emulsion with these combinations (see FIG. 2) would be similar to those shown in FIG. 1.

Surprisingly the absorbance curves in FIG. 2 are very close to each other which resulted in the SPFs of the 2 formulations to be very close, formula 1 with an in-vitro SPF (measured according to ISO 24443:2012 Determination of Sunscreen UVA Photoprotection in vitro) of 76 for Formula 1 and 70 for Formula 2. Therefore Diethylhexyl 2,6-Naphthalate can be used to replace Oxybenzone in suncare formulations.

### Formulation Examples

### Example 1 Sunscreen Lotion (O/W), Expected SPF 50+

| | | | % |
|---|---|---|---|
| Phase | Ingredient | INCI name | w/w |
| A | Emulsiphos ® | Potassium Cetyl Phosphate, Hydrogenated Palm | 2.00 |
| | | Glycerides | |
| | Neo Heliopan ® 357 | Butyl Methoxydibenzoylmethane | 2.70 |
| | Neo Heliopan ® 303 | Octocrylene | 9.00 |
| | Neo Heliopan ® OS | Ethylhexyl Salicylate | 5.00 |
| | Neo Heliopan ® HMS | Homosalate | 9.00 |
| | Neutral oil | Caprylic/Capric Triglyceride | 6.00 |
| | KP-545 | Cyclopentasiloxane, Acrylates/Dimethicone | 0.10 |
| | | Copolymer | |
| | Carbopol ® Ultrez 21 | Acrylates/C10-30 Alkyl Acrylate Crosspolymer | 1.00 |
| | Keltrol ® BT | Xanthan Gum | 0.25 |
| | Edeta ® BD | Disodium EDTA | 0.10 |
| | Corapan ® TQ | Diethylhexyl 2,6-Naphthalate | 10.00 |
| B | Aqua/Water | Aqua/Water | 49.65 |
| | Sodium Hydroxide, 10% aq. | Aqua, Sodium Hydroxide | 0.70 |
| | Soln | | |
| | Glycerin 99% | Glycerin | 3.00 |
| | SymSave ® H | Hydroxyacetophenone | 0.50 |
| | SymDiol ® 68 | 1,2-Hexanediol, Caprylyl Glycol | 0.50 |
| C | Fragrance | Parfum | 0.50 |
| | | TOTAL | 100.00 |

Manufacturing Procedure

Phase A: Heat up to approx. 85° C. without Keltrol®, when all ingredients are dissolved add Keltrol® and homogenize with an Ultra Turrax® for a short time.

Phase B: Add all ingredients to phase B and heat up to approx 80° C. and homogenize for a short time then add the hot phase B to the hot phase A and start homogenizing with an Ultra Turrax® (13000 rpm/1 minutes per 100 g emulsion). Cool down to ambient temperature while stirring and add Phase C.

Phase C: Add phase C and stir until homogeneous.

### Example 2. Antiaging Sunscreen Lotion (O/W), Expected SPF 30

| Part | Raw Materials | INCI Name | % (wt.) |
|---|---|---|---|
| A | Emulsiphos ® | Potassium Cetyl Phosphate, | 3.80 |
| | | Hydrogenated Palm Glycerides | |
| A | Lanette ® 16 | Cetyl Alcohol | 1.00 |
| A | Neo Heliopan ® 357 | Butyl | 3.00 |
| | | Methoxydibenzolymethane | |
| A | Neo Heliopan ® OS | Ethylhexyl Salicylate | 5.00 |
| A | Neo Heliopan ® 303 | Octocrylene | 8.00 |
| A | Neo Heliopan ® HMS | Homosalate | 10.00 |
| A | Corapan ® TQ | Diethylhexyl 2,6-Naphthalate | 5.00 |
| A | SymHelios ® 1031 | Benzylidene | 0.50 |
| | | Dimethoxydimethylindanone | |
| A | Silsoft ® 034 | Caprylyl Methicone | 3.00 |
| A | Dragoxat ® 89 | Ethylhexyl Isononanoate | 3.00 |
| A | Symsitive ® 1609 | Pentylene Glycol, 4-t- | 1.00 |
| | | Butylcylcohexanol | |
| A | Antaron ® WP 660 | Tricontanyl PVP | 1.00 |
| A | EDETA ® BD | Disodium EDTA | 0.10 |
| A | Copherol ® 1250 | Tocopheryl Acetete | 0.50 |
| A | Keltrol ® T | Xanthan Gum | 0.40 |
| A | Wacker-Belsil ® CDM 3526 VP | C26-28 Alkyl Dimethicone | 1.00 |
| A | Hydrolite ®-5 | Pentylene glycol | 4.25 |
| A | SymMollient ® S | Cetearyl Nonanoate | 1.00 |
| B | Water | Aqua (Water) | 40.55 |
| B | Glycerin 99% | Glycerin | 2.00 |
| C | SymSave ® H | Hydroxyacetophenone | 0.50 |
| C | Symdiol ® 68 | 1,2-Hexanediol, Caprylyl Glycol | 0.50 |
| C | Dow Corning ® 9801 Cosmetic | Dimethicone/Vinyl | 0.50 |
| | Powder | Dimethicone Crosspolymer, Silica | |
| C | Parfum | Parfum | 0.20 |
| C | Dragosantol ® 100 | Bisabalol | 0.10 |
| C | SymGlucan ® | Aqua, Glycerin, Beta-Glucan, | 1.00 |
| | | 1,2-Hexanediol, Caprylyl Glycol | |
| C | Orgasol ®Caresse | Nylon 6/12 | 2.00 |

Manufacturing Procedure

Phase A: Mix the components without TiO2 and Keltrol® T to approx. 85° C. Homogenize for a short time with an Ultra Turrax®.

Phase B: Mix the components and heat up to approx. 80° C. until dissolved. Add phase B to phase A while stirring. Cool down while stirring to 60° C. and homogenize with an Ultra Turrax®. Then cool down to ambient temperature while stirring.

Phase C: Add all ingredients step by step and stir until homogeneous with an Ultra Turrax®.

### Example 3. Low Viscosity Sunscreen Lotion (O/W), Expected SPF 50+

| Part | Raw Materials | INCI Name | % (wt.) |
|---|---|---|---|
| A | Emulsiphos ® | Potassium Cetyl Phosphate, | 0.80 |
| | | Hydrogenated Palm Glycerides | |
| A | Dacorin ® 100 SEP | Glyceryl Stearate, PEG-100 | 1.50 |
| | | Stearate | |
| A | Neo Heliopan ® 357 | Butyl | 3.00 |
| | | Methoxydibenzolymethane | |
| A | Neo Heliopan ® OS | Ethylhexyl Salicylate | 5.00 |
| A | Neo Heliopan ® 303 | Octocrylene | 10.00 |
| A | Neo Heliopan ® HMS | Homosalate | 10.00 |
| A | Corapan ® TQ | Diethylhexyl 2,6-Naphthalate | 10.00 |
| A | Neo Heliopan ® AV | Ethylhexyl Methoxycinnamate | 2.00 |
| A | SymMollient ® PDCC | Propylenediol Dicaprylate | 2.50 |
| | | Caprate | |
| A | Silsoft 034 | Caprylyl Methicone | 1.00 |
| A | Dow Corning ® Wax 2503 | Stearyl Dimethicone | 1.00 |
| A | Dow Corning ® EL 7040 Hydro | Caprylyl Methicone (and) PEG- | 2.00 |
| | Elastomer Blend | 12 Dimethicone/PPG-20 | |
| | | Crosspolymer | |
| A | EDETA ® BD | Disodium EDTA | 0.10 |
| | Prisorine ® 3505 | Isostearic Acid | 1.00 |
| A | Copherol ® 1250 | Tocopheryl Acetete | 0.50 |
| A | Keltrol ® CG SFT | Xanthan Gum | 0.15 |
| A | Pemulen ® TR 2 | Acrylates/C10-30 Alkyl Acrylate | 0.15 |
| | | Crosspolymer | |
| B | Water | Aqua (Water) | 38.60 |
| B | Glycerin 99% | Glycerin | 1.00 |
| B | Propylenglycol | Propylene Glycol | 5.00 |
| B | SymSave ® H | Hydroxyacetophenone | 0.50 |
| C | Symdiol ® 68 | 1,2-Hexanediol, Caprylyl Glycol | 1.00 |
| D | Dow Corning 9801 Cosmetic | Dimethicone/Vinyl | 1.00 |
| | Powder | Dimethicone Crosspolymer, | |
| | | Silica | |
| D | Tapioca pure | Tapioca Starch | 2.00 |
| D | Fragrance | Parfum | 0.20 |

Manufacturing Procedure

Phase A: Mix ingredients without Keltrol® and Pemulen® and heat up to approx. 85° C. When all ingredients are dissolved add Keltrol® and Pemulen® and homogenize with an Ultra Turrax® T25 for a short time (30 seconds).

Phase B: Mix ingredients and heat up to approx. 80° C. Add phase B to phase A and homogenizing with an Ultra Turrax® (13000 rpm/1 minutes per 100 g emulsion) at 60° C. Cool down to ambient temperature while stirring.

Phase C: Add to phase A/B with stirring until homogeneous.

Phase D: Add to phases A/B/C while stirring. Homogenize with an Ultra Turrax (13000 rpm/1 minutes per 100 g emulsion).

### Example 4. Infant Sunscreen Cream (O/W) without Organic UV Filters, Expected SPF 30

| Part | Raw Materials | INCI Name | % (wt.) |
|---|---|---|---|
| A | Emulsiphos ® | Potassium Cetyl Phosphate, | 1.00 |
| | | Hydrogenated Palm Glycerides | |
| A | SymMollient ® PDCC | Propylenediol Dicaprylate | 2.00 |
| | | Caprate | |
| A | Tocopherylacetat | Tocopherylacetat | 1.00 |
| A | SymMollient ® S | Cetearyl Nonanoate | 7.30 |
| A | EDTA ® BD | Disodium ETDA | 0.10 |
| A | Sweet Almond oil raff. | Prunus Amygdalus Dulcis Oil | 5.00 |
| A | Isodragol ® | Triisononanoin | 4.00 |
| A | Corapan ® TQ | Diethylhexyl 2,6-Naphthalate | 3.00 |
| A | Carnicol ® N 352 | Hydrogenated Rapeseed Oil | 3.00 |
| A | SymTio ® SP | Titanium Dioxide, Aluminum | 5.00 |
| | | Hydroxide, Cetearyl | |
| | | Nonanoate, Stearic Acid | |
| A | Keltrol ® CG-T | Xanthan Gum | 0.50 |
| B | Water dem. | Water (Aqua) | 39.85 |
| B | Zinc Oxide USP | Zinc Oxide | 20.00 |
| B | Glycerin 99.5% | Glycerin | 3.00 |
| B | SymSol ® PF-3 | Aqua, Pentylene Glycol, | 3.00 |
| | | Sodium Lauryl Sulfoacetate, | |
| | | Sodium Oleoyl Sarcosinate, | |
| | | Sodium Chloride, Disodium | |
| | | Sulfoacetate, | |
| | | Sodium Oleate, Sodium Sulfate | |
| C | Dragosantol ® 100 | Bisabolol | 0.10 |
| C | SymOcide ® PS | Phenoxyethanol, Decylene | 1.25 |
| | | Glycol, 1,2-Hexanediol | |
| C | Citric Acid pure | Citric Acid | 0.60 |
| D | Perfume oil | Parfum (Fragrance) | 0.30 |

Manufacturing Procedure.

Phase A: Heat up to approx. 85° C. without Keltrol® CG-T and Titanium Dioxide. Add Keltrol® CG-T and Titanium Dioxide and then homogenize.

Part B: Heat to 80-85° C. with stirring then add part B to part A with stirring and then homogenise.

Part C: Mix phase C together with stirring. Then add to parts A/B at about 60° C. with stirring until homogeneous. Allow to cool to room temperature and then add phase D with stirring, then homogenize.

### Example 5 Sunscreen Spray Expected SPF 50

| Part | Raw Materials | INCI Name | % (wt.) |
|---|---|---|---|
| A | Dracorin ® GOC | Glyceryl Oleate Citrate, | 2.00 |
| | | Caprylic/Capric Triglyceride | |
| A | Neo Heliopan ® 357 | Butyl Methoxydibenzoylmethane | 3.00 |
| A | Neo Heliopan ® HMS | Homosalate | 10.00 |
| A | Neo Heliopan ® 303 | Octocrylene | 10.00 |
| A | Neo Heliopan ® OS | Ethylhexyl Salicylate | 5.00 |
| A | Neo Heliopan ® AV | Ethylhexyl Methoxycinnamate | 2.00 |
| A | Corapan ® TQ | Diethylhexyl 2,6-Naphthalate | 7.50 |
| A | Dragoxat ® 89 | Diisopropyladipate | 3.00 |
| A | SymMollient ® S | Cetearyl Nonanoate | 2.00 |
| A | EDTA ® BD | Disodium EDTA | 0.10 |
| A | Copherol ® 1250 | Tocopherolacetat-Alpha | 0.50 |
| A | Silcare ® Silicone 41M65 | Stearyl Dimethicone | 1.00 |
| A | Wacker Belsil ® CMD 3526 | C26-28 Alkyl Dimethicone | 1.00 |
| | VP | | |
| A | Silsoft 034 | Caprylyl Methicone | 2.00 |
| A | Pemulen TR 2 | Acrylates/C10-30 Alkyl Acrylate | 0.20 |
| | | Crosspolymer | |
| B | Water, dest. | Water (Aqua) | 39.20 |
| B | SymSol ® PF-3 | Aqua, Pentylene Glycol, Sodium | 3.00 |
| | | Lauryl Sulfoacetate, Sodium | |
| | | Oleoyl Sarcosinate, Sodium | |
| | | Chloride, Disodium Sulfoacetate, | |
| | | Sodium Oleate | |
| B | Propylenglycol | Propylene Glycol | 5.00 |
| B | EG 56 Polymer Expert | Bis-Methoxy PEG-13 PEG- | 0.20 |
| | | 438/PPG-110 SMDI Copolymer | |
| B | SymSave ® H | Hydroxyacetophenone | 0.50 |
| C | Phenoxyethanol | Phenoxyethanol | 0.30 |
| C | SymDiol ® 68 | 1,2 Hexanediol, Caprylylglycol | 0.30 |
| C | Dow Corning ® 1503 | Dimethicone/Dimethiconol | 1.00 |
| D | Tapioca Pure | Tapioca Starch | 1.00 |
| D | Perfume oil | Fragrance (Parfum) | 0.20 |

Manufacturing Procedure.

Phase A: Mix the ingredients without Pemulen® TR2 to approx 60° C. with stirring. Add Pemulen® TR2 and homogenize for a short time, approx 0.5 min. with an Ultra Turrax® T25.

Phase B: Dissolve ExpertGel® in water while stirring. When dissolved add the rest of the ingredients and stir until a clear solution is obtained. Heat slightly if necessary to solubilize SymSave® H. Add the water phase B without stirring to the warm oil phase A. Homogenize with an Ultra Turrax® for approximately 5 min. Stir to cool down.

Phase C: Mix the ingredients stirring and then to phase A/B. Cool down while stirring.

Phase D: Add these separately to phases A/B/C with stirring at ambient temperature. Then homogenise for a short time.

### Example 6 Water Resistant Broad Spectrum O/W Expected SPF 50+

| | | | A % |
|---|---|---|---|
| Part | Ingredients | INCI | (wt.) |
| A | Emulsiphos ® | Potassium Cetyl Phosphate, | 3.50 |
| | | Hydrogenated Palm Glycerides | |
| A | Lanette ® O | Cetearylalcohol | 1.00 |
| A | Neo Heliopan ® HMS | Homosalate | 5.00 |
| A | Neo Heliopan ® 303 | Octocrylene | 10.00 |
| A | Neo Heliopan ® OS | Ethylhexyl Salicylate | 5.00 |
| A | Neo Heliopan ® 357 | Butyl Methoxydibenzoylmethane | 3.00 |
| A | Corapan ® TQ | Diethylhexyl 2,6-Naphthalate | 8.00 |
| A | Abil ® Wax 9801 | Cetyl Dimethicone | 1.00 |
| A | Silcare Silicone 41M65 | Stearyl Dimethicone | 1.00 |
| A | Baysilone ® oil PK 20 | Phenyl Trimethicone | 2.00 |
| A | SymMollient ® PDCC | Propylenediol Dicaprylate | 2.00 |
| | | Caprate | |
| A | Tocopherylacetat | Tocopheryl Acetate | 0.50 |
| A | Antaron ® V216 | VP/Hexadecene Copolymer | 0.50 |
| A | EDTA BD | Disodium EDTA | 0.10 |
| A | Keltrol ® T | Xanthan Gum | 0.50 |
| B | Water dem | Water (Aqua) | Ad 100 |
| B | SymSave ® H | Hydroxyacetophenone | 0.50 |
| B | Lara Care ® A-200 | Galactoarabinan | 0.25 |
| B | Hydrolite ® 5 | Pentylene Glycol | 3.00 |
| C | Fragrance | Fragrance (parfum) | 0.30 |

Manufacturing Procedure.

Phase A: Heat all components except for the Xanthan Gum and TiO₂ to 85° C. Then add Xanthan Gum and homogenise.

Phase B: Heat all components to 85° C. and add to Part A with stirring, stir to room temperature.

Phase C: Add Part C to Parts A & B and homogenise.

### Example 7 Sunscreen Lotion with Tanning Accelerator, Expected SPF 30

| Part | Ingredients | INCI-Name | % (wt.) |
|---|---|---|---|
| A | Emulsiphos ® | Potassium Cetyl Phosphate, | 2.00 |
| | | Hydrogenated Palm Glycerides | |
| A | Lanette 16 ® | Cetyl Alcohol | 1.00 |
| A | Neo Heliopan ® 357 | Butyl | 3.00 |
| | | Methoxydibenzoylmethane | |
| A | Neo Heliopan ® OS | Ethylhexyl Salicylate | 5.00 |
| A | Neo Heliopan ® 303 | Octocrylene | 8.00 |
| A | Neo Heliopan ® HMS | Homosalate | 5.00 |
| A | Corapan ® TQ | Diethylhexyl 2,6-Naphthalate | 5.00 |
| A | SymHelios ® 1031 | Benzylidene | 0.50 |
| | | Dimethoxydimethylindanone | |
| A | Dragoxat ® 89 | Ethylhexyl Isononanoate | 3.00 |
| A | SymMollient ® S | Cetearyl Nonanoate | 1.00 |
| A | Dow Corning ® DC 1503 | Dimethicone, Dimethiconol | 0.50 |
| A | Silcare ® Silicone 41M65 | Stearyl Dimethicone | 1.00 |
| A | Silsoft ® 034 | Caprylyl Methicone | 1.50 |
| A | EDETA ® BD | Disodium EDTA | 0.10 |
| A | Vitamin E Acetate | Tocopheryl Acetete | 0.50 |
| A | Keltrol ® T | Xanthan Gum | 0.30 |
| A | Aristoflex ® Velvet | Polyacrylate Crosspolymer-11 | 0.50 |
| B | Water dem | Aqua (Water) | 50.50 |
| C | Neo Heliopan ® Hydro | Phenylbenzimidazole Sulfonic | 1.50 |
| | | Acid | |
| C | Glycerin 99% | Glycerin | 3.00 |
| C | Dragosine | Carnosine | 0.20 |
| C | Biotive ® L-Arginine | Arginine | 1.00 |
| C | Lanette ® E | Sodium Cetearyl Sulfate | 0.70 |
| C | SymSave ® H | Hydroxyacetophenone | 0.50 |
| C | SymDiol ® 68 | 1,2 Hexanediol, Caprylylglycol | 0.50 |
| D | Fragrance | Parfum | 0.20 |
| D | Tapioca Pure | Tapioca Starch | 2.00 |
| D | SymBronze ® | Caprylic/Capric Triglyceride, | 2.00 |
| | | Isochryris Galbana Extract | |

Manufacturing Procedure.

Phase A: Mix ingredients to approx. 85° C. without Keltrol® and Aristoflex®, when all ingredients are dissolved add Keltrol® and Aristoflex® and homogenize with an Ultra Turrax® for a short time.

Part B: Mix ingredients with stirring to approximately 80° C. Add the hot phase B to the hot phase A, cool down with stirring to 60° C. and start homogenizing with an Ultra Turrax®. Cool down to ambient temperature while stirring.

Part C: Add the ingredients to parts A/B as listed with stirring and allow to cool to ambient temperature.

Part D: Add the ingredients with stirring and homogenise for a short time.

### Example 8. CC Cream with Expected SPF 50

| Part | Ingredients | INCI-Name | % (wt.) |
|---|---|---|---|
| A | Cutina ® CP | Cetyl Pamitate | 1.00 |
| A | Tegosoft MM | Myristyl Myristate | 1.00 |
| A | Lanette ® O | Cetearyl Alcohol | 1.00 |
| A | Neo Heliopan ® 357 | Butyl | 3.00 |
| | | Methoxydibenzoylmethane | |
| A | Neo Heliopan ® OS | Ethylhexyl Salicylate | 5.00 |
| A | Neo Heliopan ® HMS | Homosalate | 10.00 |
| A | Neo Heliopan ® 303 | Octocrylene | 10.00 |
| A | SymHelios ® 1031 | Benzylidene | 0.30 |
| | | Dimethoxydimethylindanone | |
| A | Corapan ® TQ | Diethylhexyl 2,6-Naphthalate | 10.00 |
| A | SymWhite ® 377 | Phenylethyl Resorcinol | 0.20 |
| A | SymRepair ® 100 | Hexyldecanol, Bisabolol, | 1.00 |
| | | Cetylhydroxyproline | |
| | | Palmitamide, Stearic Acid, | |
| | | Brassica Campestris Sterols | |
| A | Symsitive ® 1609 | Pentylene Glycol, 4-t- | 1.00 |
| | | Butylcylcohexanol | |
| A | Dragoxat ® 89 | Ethylhexyl Isononanoate | 2.00 |
| A | Silsoft(TM) 034 | Caprylyl Methicone | 3.00 |
| A | Wacker-Belsil ® CDM 3526 | C26-28 Alkyl Dimethicone | 1.00 |
| | VP | | |
| A | EDETA ® BD | Disodium EDTA | 0.10 |
| A | Copherol ® 1250 | Tocopheryl Acetete | 0.50 |
| A | Keltrol ® CG-BT | Xanthan Gum | 0.40 |
| A | Aristoflex ® Velvet | Polyacrylate Crosspolymer-11 | 0.50 |
| B | Water dem | Aqua (Water) | 31.30 |
| B | Neo Heliopan ® Hydro, | Phenylbenzimidazole Sulfonic | 1.50 |
| | | Acid | |
| B | Glycerin 99% | Glycerin | 4.00 |
| B | Dragosine ® | Carnosine | 0.20 |
| B | SymSol ® PF3 | Aqua, Pentylene Glycol, Sodium | 2.50 |
| | | Lauryl Sulfoacetate, Sodium | |
| | | Oleoyl Sarcosinate, Sodium | |
| | | Chloride, Disodium | |
| | | Sulfoacetate, Sodium Oleate | |
| B | Biotive ® L-Arginine | Arginine | 1.00 |
| B | NaOH 10% aq. | Sodium Hydroxide | 0.30 |
| B | DragoColour ® Brown | Titanium Dioxide (CI 77891), | 2.00 |
| | | Iron Oxides (CI 77492), Iron | |
| | | Oxides (CI 77491), Iron Oxides | |
| | | (CI 77499) | |
| B | SymSave ® H | Hydroxyacetophenone | 0.50 |
| C | SymDiol ® 68 | 1,2 Hexanediol, Caprylylglycol | 0.50 |
| D | Tapioca pure | Tapioca Starch | 2.00 |
| D | Orgasol ® 4000 EXD NAT | Nylon 6/12 | 2.00 |
| | COS Caresse | | |
| D | SymGlucan ® | Aqua, Glycerin, Beta-Glucan, | 1.00 |
| | | 1,2-Hexanediol, Caprylyl Glycol | |
| D | Fragrance | | 0.20 |

Manufacturing Procedure.

Phase A: Mix ingredients to approx. 85° C. without Keltrol®, Aristoflex® and titanium dioxide, when all ingredient are dissolved add Keltrol®, Aristoflex® and Titanium dioxide and homogenize with an Ultra Turrax® for a short time.

Phase B: Add the water and neutralisation agents Biotive® L-Arginine and the sodium hydroxide solution and stir until homogenious. Then add the Neo Heliopan® Hydro and stir until all has dissolved. Add the rest of ingredients without Dragocolor® to phase B and heat up to approx 80° C., add Dragocolor® and homogenize for a short time then add the hot phase B to the hot phase A and start homogenizing with an Ultra Turrax® (13000 rpm/1 minutes per 100 g emulsion). Cool down to ambient temperature while stirring.

Part C: Add the ingredients to parts A/B as listed with stirring and allow to cool to ambient temperature.

Part D: Add the ingredients with stirring and homogenise for a short time.

### Example 9. Sun Protection Sticks with Expected SPFs of (A) 30 and (B) 50+

| | | | A | B |
|---|---|---|---|---|
| | | | % | % |
| Part | Raw materials | INCI name | (w/w) | (w/w) |
| A | Neo Heliopan ® 357 | Butyl | 3.00 | 3.00 |
| | | Methoxydibenzoylmethane | | |
| A | Neo Heliopan ® 303 | Octocrylene | 3.00 | 8.00 |
| A | Neo Heliopan ® OS | Ethylhexyl Salicylate | 5.00 | 5.00 |
| A | Corapan ® TQ | Diethylhexyl 2,6-Naphthalate | 5.00 | 10.00 |
| A | Neo Heliopan ® HMS | Homosalate | 5.00 | 10.00 |
| A | SymHelios ® 1031 | Benzylidene | 0.50 | 0.50 |
| | | Dimethoxydimethylindanone | | |
| A | Copherol ® 1250 | Tocopheryl Acetate | 0.70 | 0.70 |
| A | Dracorin ® GOC | Glyceryl Oleate Citrate, | 0.50 | 0.50 |
| | | Caprylic/Capric Triglyceride | | |
| A | Lanette ® O | Cetearyl Alcohol | 7.00 | 5.00 |
| A | TeCe-Ozokerit ® N 502 | Ozokerite | 20.00 | 21.00 |
| A | Candenilla Wax LT 281 | Candelilla ((Euphorbia | 2.00 | 3.00 |
| | BI | Cerifera) Wax | | |
| A | Isoadipate | Diisopropyl Adipate | 5.00 | 2.00 |
| A | Isopropylpalmitat | Isopropyl Palmitate | 8.20 | — |
| A | PCL Liquid ® 100 | Cetearyl Ethylhexanoate | 2.00 | 5.20 |
| A | SymMollient ® S | Cetearyl Nonanoate | 6.00 | 5.00 |
| A | Wacker Belsil ® CDM | C26-28 Alkyl Dimethicone | 2.00 | 2.00 |
| | 3526 VP | | | |
| A | Silcare ® Silicone 41 | Stearyl Dimethicone | 1.00 | 1.00 |
| | M45 | | | |
| A | Neutral oil | Caprylic/Capric Triglyceride | 10.00 | — |
| A | SymMollient ® PDCC | Propylenediol Dicaprylate | 3.00 | 3.00 |
| | | Caprate | | |
| A | Dragoxat 89 | Ethylhexyl Isononanoate | 2.00 | 1.00 |
| A | Dragosantol ® 100 | Bisabolol | 0.10 | 0.10 |
| B | SymTio ® SP | Titanium Dioxide, Aluminum | 5.00 | 5.00 |
| | | Hydroxide, Cetearyl | | |
| | | Nonanoate, Stearic Acid | | |
| B | Zinc Oxide PI | Zinc Oxide | 2.00 | 5.00 |
| B | Dow Corning ® | Dimethicone/Vinyl | 2.00 | 2.00 |
| | Cosmetic Powder | Dimethicone Crosspolymer | | |
| | 9701 | (and) Silica | | |

Manufacturing Procedure.

Phase A: Mix ingredients and heat with stirring to approx. 80° C. Hold the temperature.

Phase B: Mix the ingredients then add phase B to phase A and homogenize. Hold the temperature. Stir slowly to let enclosed air escape from the mass. Transfer to the stick holders at 75-80° C.

### Example 10. Sunscreen Cream (W/O), Expected SPF 50, Water Resistant

| Part | Raw Materials | INCI Name | % (wt.) |
|---|---|---|---|
| A | Dehymuls ®PGPH | Polyglyceryl-2 | 5.00 |
| | | Dipolyhydroxystearate | |
| | Copherol ®1250 | Tocopheryl Acetate | 0.50 |
| | Permulgin ® 3220 | Ozokerite | 0.50 |
| | Aluminium stearate | Aluminium Stearate | 0.50 |
| | Neo Heliopan ® OS | Ethylhexyl Salicylate | 5.00 |
| | Neo Heliopan ® HMS | Homosalate | 15.00 |
| | Neo Heliopan ® 303 | Octocrylene | 10.00 |
| | Tegosoft ® TN | C12-15 Alkyl Benzoate | 20.00 |
| | Neo Heliopan ® 357 | Butyl | 3.00 |
| | | Methoxydibenzolymethane | |
| | EDETA ® BD | Disodium EDTA | 0.10 |
| | Corapan ® TQ | Diethylhexyl 2,6- | 10.00 |
| | | Naphthalate | |
| B | Water, dist. | Water (Aqua) | 24.60 |
| | Glycerol, 99% | Glycerin | 4.00 |
| | SymDiol ® 68 | 1,2 Hexanediol, | 0.50 |
| | | Caprylylglycol | |
| | SymSave ® H | Hydroxyacetophenone | 0.50 |
| | Magnesium sulfate | Magnesium Sulfate | 0.50 |
| C | Perfume oil | Parfum (Fragrance) | 0.30 |

Manufacturing Procedure.

Part A: Mix the ingredients with stirring at about 50° C.

Part B: Mix the ingredients with stirring at about 85° C. then add t A. Allow to cool with stirring then homogenise.

Part C: Stir in at ambient temperature.

### Example 11. Alcoholic Spray

| Phase | Ingredient | INCI name | % w/w |
|---|---|---|---|
| | Alcohol denat | Alcohol | 48.15 |
| | Neo Heliopan ® 357 | Butyl | 3.00 |
| | (622501) | Methoxydibenzoylmethane | |
| A | Neo Heliopan ® 303 | Octocrylene | 10.00 |
| | (600154) | | |
| | Neo Heliopan ® OS | Ethylhexyl Salicylate | 5.00 |
| | (131494) | | |
| | Neo Heliopan ® HMS | Homosalate | 15.00 |
| | (182573) | | |
| | Butyloctyl Salicylate | Butyloctyl Salicylate | 5.00 |
| | Corapan ® TQ | Diethylhexyl 2,6- | 10.00 |
| | (182585) | Naphthalate | |
| | KP-545 | Cyclopentasiloxane, | 1.00 |
| | | Acrylates/Dimethicone | |
| | | Copolymer | |
| | Dermacryl ® 79 | Acrylates/Octylacrylamide | 2.00 |
| | | Crosspolymer | |
| B | Fragrance | Parfum | 0.30 |
| B | Vitamin E Acetate | Tocopheryl Acetate | 0.25 |
| B | Vitamin E | Tocopherol | 0.10 |
| B | Vitamin C Palmitate | Ascorbyl Palmitate | 0.10 |
| B | Vitamin A Palmitate | Retinyl Palmitate | 0.10 |
| | | TOTAL | 100.00 |

Manufacturing Procedure.

Part A: Disperse the Dermacryl and KP-545 into the alcohol and then mix in the other ingredients with stirring at about 50° C. and coil to ambient temperature.

Part B: Mix the ingredients with stirring at ambient temperature then add to A with stirring then homogenise.

Part C: Stir in at ambient temperature

### Example 12. Antiaging Facial Lotion (O/W), Expected SPF>50

| Part | Raw Materials | INCI Name | % (wt.) |
|---|---|---|---|
| A | Glyceryl Stearate | Glyceryl Stearate | 3.00 |
| A | PEG-100 Stearate | PEG-100 Stearate | 1.00 |
| A | Neo Heliopan ® 357 | Butyl | 3.00 |
| | | Methoxydibenzolymethane | |
| A | Neo Heliopan ® OS | Ethylhexyl Salicylate | 5.00 |
| A | Neo Heliopan ® 303 | Octocrylene | 8.00 |
| A | Neo Heliopan ® HMS | Homosalate | 10.00 |
| A | Corapan ® TQ (182585) | Diethylhexyl 2,6-Naphthalate | 10.00 |
| A | SunSpheres ™ | Styrene/Acylates Copolymer | 2.00 |
| A | Silica | Silica | 5.00 |
| A | Beeswax | Cera Alba | 3.00 |
| A | KP-545 | Cyclopentasiloxane | 3.00 |
| | | Acrylates/Dimethicone | |
| | | Copolymer | |
| A | Pemulen ™ TR-2 | Acrylates/C10-30 Alkyl Acrylate | 1.00 |
| | | Crosspolymer: | |
| A | EDETA ® BD | Disodium EDTA | 0.10 |
| B | Water | Aqua (Water) | qs |
| B | Ethylhexyl Glycerin | Ethylhexyl Glycerin | 1.00 |
| B | Chlorphenesin | Chlorphenesin | 0.50 |
| B | Triethanolamine | Triethanolamine | qs |
| B | BHT | Butyl Hydroxytoluene | 0.10 |
| B | Dipotassium glycyrrizate | Dipotassium glycyrrizate | 0.10 |
| C | Dragosantol ® 100 | Bisabalol | 0.10 |
| C | SymGlucan ® | Aqua, Glycerin, Beta-Glucan, | 1.00 |
| | | 1,2-Hexanediol, Caprylyl Glycol | |
| C | Parfüm | Parfum | 0.20 |

Manufacturing Procedure

Phase A: Mix the components without Dermacyl, silica Pemulene to approx. 85° C., then add the other parts. Homogenize for a short time with an Ultra Turrax®.

Phase B: Mix the components and heat up to approx. 50° C. until dissolved. Add phase B to phase A while stirring. Cool down while stirring to 30° C. and homogenize with an Ultra Turrax®. Then cool down to ambient temperature while stirring.

Phase C: Add all ingredients step by step and stir until homogeneous with an Ultra Turrax®.

1. A composition consisting of Octisalate, Homosalate, Avobenzone, Octocrylene and, at least 3.5% Diethylhexyl 2,6-Naphthalate by weight, wherein the composition is a cosmetic, dermatological or pharmacological composition for protection of human skin and/or human hair against UV radiation and wherein the composition is free of Oxybenzone and wherein the composition maintains an SPF which is the same as a composition which consists of Octisalate, Homosalate, Avobenzone, and Octocrylene in the same concentrations but also contains oxybenzone.

2. (canceled)

3. The composition according to claim 1, wherein the composition further comprises one or more of Zinc Oxide, Titanium Dioxide, Octinoxate or Ensulizole.

4. The composition according to claim 1, wherein the composition further comprises at least one alkyl 1,2-diol with an alkyl chain length of 5 to 10.

5. The composition according to claim 1, wherein the composition further comprises Hydroxyacetophenone.

6. The composition according to claim 1, wherein the composition is a suncare formulation with an SPF of at least 30.

7. The composition according to claim 1, wherein the composition has a UVA protection factor of at least 370 nm, as measured by the Critical Wavelength Method for in vitro determination of UVA protection.

8. The composition according to claim 1, wherein the composition is an oil in water emulsion.

9. The composition according to claim 1, wherein the composition is a water in oil emulsion.

10. The composition according to claim 8, wherein the emulsion has an oil phase comprising one or more of:
a) hydrocarbon oils,
b) waxes,
c) silicone oils,
d) natural oils,
e) fatty acid esters,
f) fatty alcohols,
g) antioxidants,
i) chelating agents,
j) skin lightening agents,
k) tan accelerating agents,
l) insect repelling agents,
m) moisturizing agents, and
n) water resistant polymer.

11. The composition according to claim 8, wherein the emulsion has an aqueous phase comprising one or more of:
a) antioxidants,
b) preservation agents, and
c) chelating agents.

12. The composition according to claim 1, wherein the composition is an alcoholic spray.

13. The composition according to claim 8, wherein the composition further comprises emulsifiers selected from the group consisting of:
a) Alkyl phosphate derivatives,
b) Glyceryl oleate citrate derivatives,
c) Glyceryl stearate citrate derivatives,
d) Stearic acid esters,
e) Sorbitan esters,
f) Ethoxylated sorbitan esters,
g) Ethoxylated mono-, di- and tri glycerides, and
h) Methyl glucose esters.

14. The composition according to claim 13, wherein the amount of emulsifier is in the range of 1 to 10% by weight, related to the composition.

15. The composition according to claim 1, wherein the composition is a dermatological active composition.

16. A method for obtaining a composition according to claim 1, comprising the following steps:
(a) providing at least one oil phase comprising Diethylhexyl 2,6-Naphthalate;
(b) providing at least one aqueous phase;
wherein said oil phase and said aqueous phase are free of oxybenzone;
(c) heating and homogenizing each of said oil phase and said aqueous phase separately;
(d) adding said aqueous phase to said oil phase to obtain a mixture; and
(e) homogenizing said mixture.

17. The method of claim 15, wherein the phases before step (c) are heated to temperatures between 70 and 90° C.

18. The composition according to claim 9, wherein the emulsion has an oil phase comprising one or more of:
a) hydrocarbon oils,
b) waxes,
c) silicone oils,
d) natural oils,
e) fatty acid esters,
f) fatty alcohols,
g) antioxidants,
i) chelating agents,
j) skin lightening agents,
k) tan accelerating agents,
l) insect repelling agents,
m) moisturizing agents, and
n) water resistant polymer.

19. The composition according to claim 9, wherein the emulsion has an aqueous phase comprising one or more of:
a) antioxidants,
b) preservation agents, and
c) chelating agents.

20. The composition according to claim 9, wherein the composition further comprises emulsifiers selected from the group consisting of:
i) Alkyl phosphate derivatives,
j) Glyceryl oleate citrate derivatives,
k) Glyceryl stearate citrate derivatives,
l) Stearic acid esters,
m) Sorbitan esters,
n) Ethoxylated sorbitan esters,
o) Ethoxylated mono-, di- and tri glycerides, and
p) Methyl glucose esters.
